SEQUENCE LISTING (30 sequences)

SEQ ID NO: 1 - protein, 9 aa, synthetic construct
  REGION 1..9: note = Linker 1
GGSGGGSGG

SEQ ID NO: 2 - protein, 13 aa, synthetic construct
  REGION 1..13: note = Linker 2
SGGGGGDKTHTCP

SEQ ID NO: 3 - protein, 12 aa, synthetic construct
  REGION 1..12: note = Linker 3
GSAGSAAGSGEF

SEQ ID NO: 4 - protein, 220 aa, synthetic construct
  REGION 1..220: note = Knob part
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 220 aa, synthetic construct
  REGION 1..220: note = Hole part
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 10 aa, synthetic construct
  REGION 1..10: note = Antigenic peptide - NS5
KSEYMTSWFY

SEQ ID NO: 7 - protein, 9 aa, synthetic construct
  REGION 1..9: note = Antigenic peptide - NS4B
LLWNGPMAV

SEQ ID NO: 8 - protein, 9 aa, synthetic construct
  REGION 1..9: note = Antigenic peptide - NS3
VLWDIPTPK

SEQ ID NO: 9 - protein, 9 aa, synthetic construct
  REGION 1..9: note = Antigenic peptide - NS3
IYGIFQSTF

SEQ ID NO: 10 - protein, 9 aa, synthetic construct
  REGION 1..9: note = Antigenic peptide NS5
RPIDDRFGL

SEQ ID NO: 11 - protein, 9 aa, synthetic construct
  REGION 1..9: note = Antigenic peptide NS3
SVKEDLVAY

SEQ ID NO: 12 - protein, 10 aa, synthetic construct
  REGION 1..10: note = Antigenic peptide - NS2A
HAVPFGLVSM

SEQ ID NO: 13 - protein, 9 aa, synthetic construct
  REGION 1..9: note = Antigenic peptide - NS2B
HPFALLLVL

SEQ ID NO: 14 - protein, 10 aa, synthetic construct
  REGION 1..10: note = Antigenic peptide - E
TESWIVDRQW

SEQ ID NO: 15 - protein, 9 aa, synthetic construct
  REGION 1..9: note = Antigenic peptide - NS1
KTWGKNLVF

SEQ ID NO: 16 - protein, 248 aa, synthetic construct
  REGION 1..248: note = scFv - CD19
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTV

SEQ ID NO: 17 - protein, 253 aa, synthetic construct
  REGION 1..253: note = scFv - 10-1074
SYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGGGGSGGGGSGGGGSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTV

SEQ ID NO: 18 - DNA, 6234 bp, synthetic construct
  misc_feature 1..6234: note = scFv CD19 in pcDNA3.1
  source 1..6234: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccgccaccatggacatgcgcgtacccgcccagctgttgggtctcttgctcctttggttgtcaggagcccgatgcgacatccaattgactcaaagtccagcatctttggctgtctcccttggtcaaagagctaccatctcatgcaaagctagtcagagcgtcgattacgatggagattcatatctgaactggtatcagcagatccctgggcagcccccaaaacttcttatctatgatgcctcaaaccttgtgtcaggcatacctcccagattcagcggatctggatcagggactgattttactttgaatatccacccggtagaaaaggttgacgcggcgacgtaccactgccaacaatccactgaggatccctggacctttggcggcggcaccaaactggaaatcaaaggagggggcggttctggaggtggaggtagcggcggaggaggttctcaagtacagctgcaacaatcaggggcggagttggtccggccagggagtagtgttaagatctcatgtaaggcgtccggctacgctttcagctcctattggatgaactgggttaaacaaaggccaggtcaggggcttgagtggataggccagatctggcccggtgatggtgatacaaattataacggcaagtttaaagggaaagcgaccctgaccgctgacgaatcttcaagtacagcctacatgcaactcagcagtctcgcgtctgaagattcagccgtttacttctgcgcgaggcgcgagaccactacggtgggccgatattactacgccatggactactggggtcaaggtaccaccgttacggtcagctctggaagtgggctgaatgacatatttgaagcgcagaaaatagagtggcatgaataattctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 19 - DNA, 6267 bp, synthetic construct
  misc_feature 1..6267: note = scFv 10-1074 in pcDNA3.1
  source 1..6267: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccgccaccatggatatgagagttcctgctcagttgctgggcctgctgctgctttggctgagcggggcaaggtgctcatacgttcgcccactgtcagttgctctgggtgaaacagcgagaatatcctgcggtcgacaagcgctcgggtctagggccgtacaatggtatcagcaccgccccggtcaggcgccaatcctcctgatatataacaaccaagaccggccttcaggaatacctgaacgattctctggaacaccggatataaacttcggtactcgggcgacactcacaatttctggagttgaggctggcgacgaggccgactactactgccacatgtgggactcacgatcaggcttctcttggtcctttggaggggccacaagacttacagttctgggcggtggtggttccggaggtgggggatctggaggcggggggtcccaagttcaacttcaggagagtggaccagggttggtcaagccgtccgagactcttagtgtaacatgcagtgtttccggcgatagcatgaacaattattactggacttggattcggcagagtcccggaaagggcttggagtggataggctacatttccgatcgagaatctgcaacctataacccaagtctcaattccagagtcgtcatttcacgagacactagtaaaaatcaacttagtctgaaacttaatagtgttacaccggccgacaccgccgtctactattgcgccaccgcgcgacggggtcagcggatttacggagttgtaagctttggggaattcttctattattactcaatggatgtctggggaaaggggacaaccgtaactgtcagtagcggctccggactcaacgacattttcgaagcccaaaagattgagtggcatgagtaattctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 20 - protein, 559 aa, synthetic construct
  REGION 1..559: note = Knob-into-Hole (no ScFv) - Knob
MDMRVPAQLLGLLLLWLSGARCGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWSGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGKGGGGSGLNDIFEAQKIEWHEHHHHHHHHHH

SEQ ID NO: 21 - protein, 351 aa, synthetic construct
  REGION 1..351: note = Knob-into-Hole (no ScFv) - Hole
MDMRVPAQLLGLLLLWLSGARCLLWNGPMAVGGGGSGGGGSGGGGSIQRTPKIQVYSRHPAENGKSNFLNCYVSGFHPSDIEVDLLKNGERIEKDEYACRVNHVTLSQPKIVKWDRDMSGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 808 aa, synthetic construct
  REGION 1..808: note = Knob-into-Hole - Knob1
MDMRVPAQLLGLLLLWLSGARCGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWSGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGKGGSGGGSGGSYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGGGGSGGGGSGGGGSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSGGGGSHHHHHHHHHH

SEQ ID NO: 23 - protein, 812 aa, synthetic construct
  REGION 1..812: note = Knob-into-Hole - Knob2
MDMRVPAQLLGLLLLWLSGARCGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWSGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGKSGGGGGDKTHTCPSYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGGGGSGGGGSGGGGSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSGGGGSHHHHHHHHHH

SEQ ID NO: 24 - protein, 811 aa, synthetic construct
  REGION 1..811: note = Knob-into-Hole - Knob3
MDMRVPAQLLGLLLLWLSGARCGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWSGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGKGSAGSAAGSGEFSYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGGGGSGGGGSGGGGSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSGGGGSHHHHHHHHHH

SEQ ID NO: 25 - protein, 615 aa, synthetic construct
  REGION 1..615: note = Knob-into-Hole - Hole1
MDMRVPAQLLGLLLLWLSGARCLLWNGPMAVGGGGSGGGGSGGGGSIQRTPKIQVYSRHPAENGKSNFLNCYVSGFHPSDIEVDLLKNGERIEKDEYACRVNHVTLSQPKIVKWDRDMSGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGKGGSGGGSGGSYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGGGGSGGGGSGGGGSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSS

SEQ ID NO: 26 - protein, 619 aa, synthetic construct
  REGION 1..619: note = Knob-into-Hole - Hole2
MDMRVPAQLLGLLLLWLSGARCLLWNGPMAVGGGGSGGGGSGGGGSIQRTPKIQVYSRHPAENGKSNFLNCYVSGFHPSDIEVDLLKNGERIEKDEYACRVNHVTLSQPKIVKWDRDMSGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGKSGGGGGDKTHTCPSYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGGGGSGGGGSGGGGSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSS

SEQ ID NO: 27 - protein, 618 aa, synthetic construct
  REGION 1..618: note = Knob-into-Hole - Hole3
MDMRVPAQLLGLLLLWLSGARCLLWNGPMAVGGGGSGGGGSGGGGSIQRTPKIQVYSRHPAENGKSNFLNCYVSGFHPSDIEVDLLKNGERIEKDEYACRVNHVTLSQPKIVKWDRDMSGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGKGSAGSAAGSGEFSYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGGGGSGGGGSGGGGSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSS

SEQ ID NO: 28 - DNA, 13639 bp, synthetic construct
  misc_feature 1..13639: note = pBR-HIV-1 M NL4-3 R5
  source 1..13639: mol_type = other DNA
atttttccacatgttaaaattttctgtcacatttaccaatactacttcttgtgggttggggtctgtgggtacacaggcatgtgtggcccaaacattatgtacctctgtatcatatgctttagcatctgatgcacaaaatagagtggtggttgcttccttccacacaggtaccccataatagactgtgacccacaatttttctgtagcactacagatcatcaatatcccaaggagcatggtgccccatttccacccccatctccacaagtgctgatacttctccttcactctcattgccactgtcttctgctctttctattagtctatcaattaacctgtctatttttctttgtcttaatattttcctatattctatgattactatggaccacacaactattgctattattattgctactactaatgctactattgctactattataggttgcattacatgtactacttactgctttgatagagaagcttgatgagtctgactgttctgatgagctcttcgtcgctgtctccgcttcttcctgccataggagatgcctaaggcttttgtcatgaaacaaacttggcaatgaaagcaacactttttacaatagcaattggtacaagcagttttaggctgacttcctggatgcttccagggctctagtctaggatctactggctccatttcttgctctcctctgtcgagtaacgcctattctgctatgtcgacacccaattctgaaatggataaacagcagttgttgcagaattcttattatggcttccactcctgcccaagtatccccgtaagtttcatagatatgttgtcctaagttatggagccatatcctaggaaaatgtctaacagcttcactcttaagttcctctaaaagctctagtgtccattcattgtatggctccctctgtggcccttggtcttctggggcttgttccatctgtcctctgtcagtttcctaacactaggcaaaggtggctttatctgttttggttttattaatgctgctagtgccaagtactgtagagatcctaccttgttatgtcctgcttgatattcacacctaggactaactatacgtcctaatatggtatttcttatagcagattctgaaaaacaatcaaaatagtgcagatgaattagttggtctgctaggtcagggtctacttgtgtgctatatctctttttcctccattctatggagactccctgacccaaatgccagtctctttctcctgtatgcagaccccaatatgttgttattactaatttagcatcccctagtgggatgtgtacttctgaacttatttttggattagtactttcatagtgatgtctataaaaccagtccttagctttccttgaaatatacatatggtgttttactaatcttttccatgtgttaatcctcatcctgtctacttgccacacaatcatcacctgccatctgttttccataatccctgatgatctttgcttttcttcttggcactacttttatgtcactattatcttgtattactactgccccttcacctttccagaggagctttgctggtcctttccaaactggatctctgctgtccctgtaataaacccgaaaattttgaatttttgtaatttgtttttgtaattctttagtttgtatgtctgttgctattatgtctactattctttcccctgcactgtaccccccaatccccccttttcttttaaaattgtggatgaatactgccatttgtactgctgtcttaagatgttcagcctgatctcttacctgtcctataattttctttaattctttattcatagattctattactccttgactttggggattgtagggaatgccaaattcctgcttgatccccgcccaccaacaggcggccttaactgtagtactggtgaaattgctgccattgtctgtatgtactgtttttactggccatcttcctgctaattttaagaggaagtatgctgtttcttgccctgtctctgctggaattacttctgcttctatatatccactggctacatgaactgctaccaagataacttttccttctaaatgtgtacaatctagctgccatattcctgggctacagtctacttgtccatgcatggcttccccttttagctgacatttatcacagctggctactatttcttttgctactacaggtggtaggttaaaatcactagccattgctctccaattactgtgatatttctcatgttcttcttgggccttatctattccatctaaaaatagtactttcctgattccagcactgaccaatttatctacttgttcatttcctccaattcctttgtgtgctggtacccatgccaggtagactttttccttttttattaactgctctattatttgactgactaactctgattcactcttatctggttgtgcttgaatgattcccaatgcatattgtgagtctgtcactatgtttacttctaatcccgaatcctgcaaagctagatgaattgcttgtaactcagtcttctgatttgttgtgtccgttagggggacaactttttgtcttcctctgtcagttacatatcctgcttttcctaatttagtttccctattggctgccccatctacatagaaagtttctgctcctattatgggttctttctctaactggtaccataacttcactaagggaggggtattgacaaactcccactcaggaatccaggtggcttgccaatactctgtccaccatgcttcccatgtttccttttgtatgggtaatttaaatttaggagtctttccccatattactatgctttctgtggctattttttgtactgcctctgttaattgtttcacatcattagtgtgggcacccttcattcttgcatactttcctgttttcagatttttaaatggctcttgataaatttgatatgtccattggccttgcccctgcttctgtatttctgctattaagtcttttgatgggtcataatacactccatgtaccggttcttttagaatctccctgttttctgccagttctagctctgcttcttctgttagtggtactacttctgttagtgctttggttcccctaagaagtttacataattgccttactttaatccctgcataaatctgacttgcccaattcaattttcccactaatttctgtatgtcattgacagtccagctgtccttttctggcagcactataggctgtactgtccatttatcaggatggagttcataacccatccaaaggaatggaggttctttctgatgttttttgtctggtgtggtaaatccccacctcaacagatgttgtctcagttcctctatttttgttctatgctgccctatttctaagtcagatcctacatacaaatcatccatgtattgatagatgactatgtctggattttgttttctaaaaggctctaagatttttgtcatgctacactggaatattgctggtgatcctttccatccctgtggaagcacattgtactgatatctaatccctggtgtctcattgtttatactaggtatggtaaatgcagtatacttcctgaagtctttatctaagggaactgaaaaatatgcatcgcccacatccagtactgttactgattttttctgttttaaccctgcaggatgtggtattcctaattgaacttcccagaaatcttgagttctcttattaagttctctgaaatctactaattttctccatttagtactgtcttttttctttatggcaaatactggagtattgtatggattttcaggcccaatttttgaaatttttccttccttttccatttctgtacaaatttctactaatgcttttattttttcttctgtcaatggccattgtttaacttttgggccatccattcctggctttaattttactggtacagtctcaataggactaatgggaaaatttaaagtgcagccaatctgagtcaacagatttcttccaattatgttgacaggtgtaggtcctactaatactgtacctatagctttatgtccgcagatttctatgagtatctgatcatactgtcttactttgataaaacctccaattccccctatcatttttggtttccatcttcctggcaaattcatttcttctaatactgtatcatctgctcctgtatctaatagagcttcctttaattgcccccctatctttattgtgacgaggggtcgctgccaaagagtgatctgagggaagctaaaggatacagttccttgtctatcggctcctgcttctgagagggagttgttgtctcttccccaaacctgaagctctcttctggtggggctgttggctctggtctgctctgaagaaaattccctggccttcccttgtgggaaggccagatcttccctaaaaaattagcctgtctctcagtacaatctttcatttggtgtccttcctttccacatttccaacagccctttttcctaggggccctgcaatttttggctatgtgcccttctttgccacaattgaaacacttaacagtctttctttggttcctaaaattgcctttctgtatcattatggtagctggatttgttacttggctcattgcttcagccaaaactcttgctttatggccgggtccccccactccctgacatgctgtcatcatttcttctagtgtcgctcctggtcccaatgcttttaaaatagtcttacaatctgggttcgcattttggaccaacaaggtttctgtcatccaattttttacctcttgtgaagcttgctcggctcttagagttttatagaatcggtctacatagtctctaaagggttcctttggtccttgtcttatgtccagaatgctggtagggctatacattcttactattttatttaatcccaggattatccatcttttatagatttctcctactgggataggtggattatgtgtcatccatcctatttgttcctgaagggtactagtagttcctgctatgtcacttccccttggttctctcatctggcctggtgcaataggccctgcatgcactggatgcaatctatcccattctgcagcttcctcattgatggtctcttttaacatttgcatggctgcttgatgtccccccactgtgtttagcatggtatttaaatcttgtggggtggctccttctgataatgctgaaaacatgggtattacttctgggctgaaagccttctcttctactacttttacccatgcatttaaagttctaggtgatatggcctgatgtaccatttgcccctggaggttctgcactatagggtaattttggctgacctggctgttgtttcctgtgtcagctgctgcttgctgtgcctttttcttacttttgttttgctcttcctctatcttatctaaggcttccttggtgtcttttacatctatcctttgatgcacacaatagaggactgctattgtattatataatgatctaagttcttctgatcctgtctgaagggatggttgtagctgtcccagtatttgtctacagccttctgatgtctctaaaaggccaggattaactgcgaatcgttctagctccctgcttgcccatactatatgttttagtttatattgtttctttccccctggccttaaccgaattttttcccatttatctaattctcccccgcttaataccgacgctctcgcacccatctctctccttctagcctccgctagtcaaaatttttggcgtactcaccagtcgccgcccctcgcctcttgccgtgcgcgcttcagcaagccgagtcctgcgtcgagagatctcctctggctttactttcgctttcaagtccctgttcgggcgccactgctagagattttccacactgactaaaagggtctgagggatctctagttaccagagtcacacaacagacgggcacacactactttgagcactcaaggcaagctttattgaggcttaagcagtgggttccctagttagccagagagctcccaggctcagatctggtctaaccagagagacccagtacaggcaaaaagcagctgcttatatgtagcatctgagggctcgccactccccagtcccgcccaggccacacctccctggaaagtccccagcggaaagtcccttgtagaaagctcgatgtcagcagtctttgtagtactccggatgcagctctcgggccacgtgatgaaatgctaggcggctgtcaaacctccactctaacacttctctctcagggtcatccattccatgcaggctcacagggtgtaacaagctggtgttctctcctttattggcctcttctaccttatctggctcaactggtactagcttgtagcaccatccaaaggtcagtggatatctgacccctggccctggtgtgtagttctgccaatcagggaagtagccttgtgtgtggtagatccacagatcaaggatatcttgtcttctttgggagtgaattagcccttccaacgcgcgcgtagcgctgtcgacaagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcgcgagcggccgctctagaactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaggcaaaaagcagctgcttatatgcagcatctgagggctcgccactccccagtcccgcccaggccacgcctccctggaaagtccccagcggaaagtcccttgtagcaagctcgatgtcagcagttcttgaagtactccggatgggatatcttgtcttctttgggagtgaattagcccttccagtccccccttttcttttaaaaagtggctacccgggttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgacgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctagccatggtattatcatcgtgtttttcaaaggaaaaccacgtccccgtggttcggggggcctagacgttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggcggaattcgcgtcagcagtctttgtagtactccgggatccgacgcgttcagcagttcttgaagtactccggatgcagctctcgggccacgtgatgaaatgctaggcggctgtcaaacctccactctaacacttctctctcagggtcatccattccatgcaggctcacagggtgtaacaagctggtgttctctcctttattggcctcttctaccttatctggctcagctggtactagcttgtagcaccatccaaaggtcagtggatatctgacccctggccctggtgtgtagttctgccaatcagggaagtagccttgtgtgtggtagatccacagatcaaggatatcttgccttctttgggagtgaattagcccttccagtccccccttttcttttaaaaagtggctaagatctacagctgccttgtaagtcattggtcttaaaggtacctgaggtgtgactggaaaacccacctcttcctcctcttgtgcttctagccaggcacaagcagcattgttagctgctgtattgctacttgtgattgctccatgtttttctaggtctcgagatactgctcccaccccatctgctgctggctcagctcgtctcattctttcccttacagcaggccatccaatcacactactttttgaccacttgccacccatcttatagcaaaatcctttccaagccctgtcttattcttctaggtatgtggcgaatagctctataagctgcttgtaatacttctataaccctatctgtcccctcagctactgctatggctgtggcattgagcaagttaacagcactattctttagttcctgactccaatactgtaggagattccaccaatatttgagggcttcccaccccctgcgtcccagaagttccacaatcctcgttacaatcaagagtaagtctctcaagcggtggtagctgaagaggcacaggctccgcagatcgtcccagataagtgctaaggatccgttcactaatcgaatggatctgtctctgtctctctctccaccttcttcttctattccttcgggcctgtcgggtcccctcgggattgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagcttgtgtaattgttaatttctctgtcccactccatccaggtcatgttattccaaatctgttccagagatttattactccaactagcattccaaggcacagcagtggtgcaaatgagttttccagagcaaccccaaatccccaggagctgttgatcctttaggtatctttccacagccaggattcttgcctggagctgtttgatgccccagactgtgagttgcaacagatgctgttgcgcctcaatagccctcagcaaattgttctgctgctgcactatatcagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatcgcctcctccaggtctgaagatctcggacccattgttgttattaccaccatctcttgttaatagcagcccagtaatatttgatgaacatctaatttgtccactgatgggaggggcatacattgcttttcctacttcctgccacatgtttataaattgttttattctgcatgggagtgtgattgtgtcacttccttcagtgttatttgacccttcagtactccaagtactattaaaccaagtactattaaacagttgtgttgaattacagtagaaaaattcccctccacaattaaaactgtgcgttacaatttctgggtcccctcctgaggattgcttaaagattattgttttattatttccaaattgttctcttaatttgctagctatctgttttaaagtggcattccattttgctctactaatgttacaatgtgcttgtcttatatctcctattatttgtcctgttgtataccatgctcgccctggtcctagatgtatactttttcttgtattgttgttgggtcttgtacaattaatttctacagatgtgttcagctgtactattatggttttagcattgtctgtgaaattggcagatctaattactacatcttcttctgctagactgccatttaacagcagttgagttgatactactggcctgattccatgtgtacattgtactgtgctgacatttgtacatggtcctgttccattgaacgtcttattattacattttagaatcgcaaaaccagccggggcacaataatgtatgggaattggctcaaaggatacctttggacaggcctgtgtaatgactgaggtgttacaacttatcaacctatagctggtattatctattggtactatatcaagtttataaaagaatgcatattctttctgcaccttatctcttatgcttgtgctgatattgaaagagcagttttttatctctcctttctccattatcattctcccgctactactattggtattagtatcattcttcaaatcagtgcactttaaactaacacagagtggggttaattttacacatggctttaggctttgatcccataaactgattatatcctcatgcatctgttctaccatgtc

SEQ ID NO: 29 - protein, 289 aa, synthetic construct
  REGION 1..289: note = encoded CD19 from pcDNA3.1
MDMRVPAQLLGLLLLWLSGARCDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGSGLNDIFEAQKIEWHE

SEQ ID NO: 30 - protein, 294 aa, synthetic construct
  REGION 1..294: note = encoded 10-1074 from pcDNA3.1
MDMRVPAQLLGLLLLWLSGARCSYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGGGGSGGGGSGGGGSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSGSGLNDIFEAQKIEWHE

(19) United States
(12) Patent Application Publication
(10) Pub. No.: US 2025/0059254 A1
(43) Pub. Date: Feb. 20, 2025

(54) PROTEINACEOUS COMPOUND FOR GENERATING SPECIFIC CYTOTOXIC T-CELL EFFECT

(71) Applicants: Aarhus Universitet, Aarhus C (DK); Region Midtjylland, Viborg (DK)

(72) Inventors: Martin Tolstrup, Aarhus C (DK); Mariane Høgsbjerg Schleimann, Aarhus C (DK); Christina Valbirk Konrad, Aarhus C (DK); Jesper Damsgaard Gunst, Aarhus C (DK)

(21) Appl. No.: 18/724,341

(22) PCT Filed: Jan. 3, 2023
(86) PCT No.: PCT/EP2023/050069

§ 371 Date: Jun. 26, 2024

(30) Foreign Application Priority Data
Jan. 3, 2022 (EP) 22150048.1

(51) Int. Cl. C07K 14/74; C07K 16/10; C07K 16/28

(52) U.S. Cl. CPC C07K 14/70539; C07K 16/1063; C07K 16/2803; C07K 2317/622; C07K 2317/73; C07K 2317/76; C07K 2319/00

(57) ABSTRACT

The present invention relates to a proteinaceous compound comprising a first binding moiety, such as an antibody or fragment thereof, having affinity for a target of interest, and a pMHC comprising an MHC molecule, such as an HLA molecule, presenting an antigenic peptide, wherein the presented antigenic peptide has affinity for CD8+ T-cells induced by the antigenic peptide, such as induced by vaccination. The present invention further relates to the proteinaceous compound for use as a medicament. Also, the present invention relates to the proteinaceous compound for treatment of cancer and for treating of a subject infected with a pathogen.

### TECHNICAL FIELD OF THE INVENTION

The present invention relates to a proteinaceous compound able to targeting target cells of interest such as cancer cells or cells infected by pathogens. Also, the present invention relates to a proteinaceous compound for treating cancer and a subject infected with a pathogen.

### BACKGROUND OF THE INVENTION

Diseases in which the immune system responds inadequately are very prevalent. In adaptive immune surveillance, cytotoxic CD8+ T cells are educated to kill cells that express the T cell's cognate antigen in the context of Human Leukocyte Antigen (HLA) presentation. That means one CD8+ T cell recognizes one antigen, which is a very efficient and highly specific immune control mechanism. However, in a number of diseases, recognition of antigen can be impaired by mutational resistance or via HLA I downregulation both of which support escape from CD8+ T cell killing. Therefore, in chronic disease settings CD8+ T cells often loose potency due to the development of immune escape or as a consequence of immune exhaustion. Both scenarios mean that antigen-specific CD8+ T cells provide inadequate immune surveillance and killing of diseased cells. Novel developments in immunotherapy, such as the development of Chimeric Antigen Receptor (CAR) T cells, have revolutionized precision technology medicine in e.g. cancer treatment by mitigating some of these losses of potency mechanism. However, these technologies are still very expensive and require highly advanced ex vivo cell manipulations.

Immunotherapy focuses to harness the power of immune effector cells and CD8+ T cells in particular. The development of modified antibody-like-molecules capable of cross-linking diseased cells and T cells is one strategy that has successfully augmented T cell responses against tumorantigen and diseased cells. An example is the Bispecific T cell engager antibodies (BiTE), molecules that link the single chain variable fragment (scFv) from an antibody specific for a disease-marking antigen with the scFv of a CD3-specific antibody. For example, blinatumomab bridging CD19 and CD3 was approved by the US FDA for treating refractory Acute Lymphoblastic Leukemia in 2014.

For most of the bispecific technologies, anti-CD3 binding scFv are used, which means these drugs broadly engage all T lymphocytes independent of lineage commitment. This non-discriminative engagement of T cells may not be the most effective way of harnessing T cell function as the broad activation often results in excessive immune inflammation and can compromise effective immune surveillance by diverting T cells towards the chosen target irrespective of their functional characteristic and differentiation state.

A second major focus area in immunotherapy is the development of CAR-T cells. This technology relies on ex vivo manipulation of cells to design killer T cells that express an antigen binding moiety (e.g. scFv) linked to T cell signaling motifs, thus enabling the genetically modified T cell to kill antigen-expressing target cells. CAR-T cells depend on ex vivo expansion followed by transplantation of large numbers of modified T cells. Thus, strict production requirements must be in place as transduction ex vivo of a leukemic cell recently led to a CAR-T cell resistant leukemic clone that repopulated the patient following infusion.

Hence, an improved immunotherapy would be advantageous, and in particular a more efficient and/or reliable immunotherapy would be advantageous.

### SUMMARY OF THE INVENTION

The present invention operates by linking the target cells and the CD8+ T-cells (effector cells). Hereby directing the cytotoxic effect of the CD8+ T-cell towards the target cells inducing their destruction. Thus, the present invention holds several advantages:
no ex vivo expansion of T cells is needed, why the risk of generating cancerous clone populations is avoided;
high amounts of autologous CD8+ T-cells are “activated”
active T-cells are generated by vaccination, which is a cheap approach as compared to e.g. the CAR-T technique;
treatment can be stopped at any time if side effect occurs, since stopping to provide the proteinaceous compound would “just” leave the patient with protection against the disease of the vaccination e.g. Yellow Fever;
only CD8+ T-cells and not all T lymphocytes independent of lineage commitment are engaged
the compound is highly efficient to produce and very stable.

Thus, an object of the present invention relates to the provision of an immunotherapy compound able to selectively activate only antigen-specific immune killer cells while leaving the naive surveilling immune cells untouched. A further object of the present invention is the provision of an efficacious technology and immunotherapy with less toxicity and more scalable production requirements that solves the mentioned problems of the prior art.

Thus, one aspect of the invention relates to a proteinaceous compound comprising:
a) a first binding moiety, such as an antibody or fragment thereof, having affinity for a target of interest, and
b) a pMHC comprising an MHC molecule, such as an HLA molecule, presenting an antigenic peptide, wherein the presented antigenic peptide has affinity for CD8+ T-cells induced by the antigenic peptide, such as induced by vaccination.

Evidently, a further aspect of the invention thus relates to a proteinaceous compound (1) comprising:
a) a first binding moiety (3), such as an antibody or fragment thereof, having affinity for a target of interest (5), and
b) a pMHC comprising an MHC molecule (7), such as an HLA molecule, presenting an antigenic peptide (9) derived from Yellow Fever Virus (YFV), wherein the presented antigenic peptide has affinity for CD8+ T-cells (11) induced by the antigenic peptide (9), such as induced by vaccination.

In particular, an aspect of the invention relates to a proteinaceous compound (1) comprising:
a) a first binding moiety (3), such as an antibody or fragment thereof, having affinity for a target of interest (5), and
b) a pMHC comprising an MHC molecule (7), such as an HLA molecule, presenting an antigenic peptide (9), wherein the presented antigenic peptide has affinity for CD8+ T-cells (11) induced by the antigenic peptide (9), such as induced by vaccination,
   wherein the compound (1) comprises:
1) a first subunit (17) comprising said antigenic peptide (9) of the pMHC and β2-microglobulin, and
2) a second subunit (19) comprising said MHC molecule (7) without the β2-microglobulin, said first subunit (17) and said second subunit (19) being linked via a heterodimerization domain.

Another aspect of the present invention relates to a pharmaceutical composition comprising the proteinaceous compound as described herein.

Yet another aspect of the present invention relates to a combination comprising
the proteinaceous compound as described herein; and
a vaccine, wherein said vaccine is for inducing CD8+ T-cells directed against the antigenic peptide.

Still another aspect of the present invention relates to a proteinaceous compound as described herein or a pharmaceutical composition as described herein for use as a medicament, in the treatment of a subject, wherein the subject has previously received a vaccine for inducing CD8+ T-cells directed against the antigenic peptide; and wherein the first binding moiety, having affinity for a target of interest,
has affinity for an epitope of the pathogen causing the disease, such as a virus; or
has affinity for a cell surface marker of a cancer.

Still another aspect of the present invention relates to a proteinaceous compound as described herein or a pharmaceutical composition as described herein, for use in the treatment of a subject infected with a pathogen, such as HIV, wherein the subject has previously received a vaccine for inducing CD8+ T-cells directed against the antigenic peptide;

wherein the first binding moiety, having affinity for a target of interest, has affinity for an epitope of the pathogen causing the disease, such as a virus, such as HIV, like HIV-1 or HIV-2.

Still another aspect of the present invention relates to a proteinaceous compound as described herein or the pharmaceutical composition as described herein for use in the treatment of a cancer, wherein the subject has previously received a vaccine for inducing CD8+ T-cells directed against the antigenic peptide; wherein the first binding moiety, having affinity for a target of interest, has affinity for a cell surface marker of the cancer.

Still another aspect of the present invention relates to a vaccine suitable for inducing CD8+ T-cells directed against an antigenic peptide, for use in the treatment of a subject
infected with an intracellular pathogen, such as a virus, such as HIV; or
suffering from cancer,
   wherein the subject is scheduled for treatment with the proteinaceous compound as described herein or a pharmaceutical composition as described herein after treatment with the vaccine.

Still further aspect of the disclosure relates to a nucleic acid encoding the proteinaceous compound (1) as described herein.

As such, also an aspect of the present disclosure relates to a vector comprising the nucleic acid.

Still another aspect of the present invention relates to a cell comprising
the vector as described herein; or
two or more vectors who in combination encode the proteinaceous compound (1) as described herein.

### BRIEF DESCRIPTION OF THE FIGURES

FIG. 1 shows construction and principle of a proteinaceous compound. (A) Illustration of the principle of the proteinaceous compound technology. A potent response of YF-epitope-specific CD8+ T cells is generated by YF-17D vaccination, which is redirected towards a target of interest by introduction of the proteinaceous compound. (B) Close-up of an embodiment of a proteinaceous compound bound to target cell and effector cell (CD8+ T cell).

FIG. 2 shows functional assessment of a proteinaceous compound. (A) Western blots with anti-AviTag or Streptavidin-HRP showing successful expression and biotinylation of scFv-10-1074 and scFv-CD19 proteins from transient transfection of mammalian cells with pcDNA3.1(+) expression plasmids encoding scFv. Upper panel: CC=Cell control, M=Mock, S1=scFv-10-1074, S2=scFv-CD19. Lower panels: S=scFv, −Sb=scFv-biotin removed, eSb=eluted scFv-biotin. (B) Functional assessment of the target cell binding capacity of scFv-10-1074 and scFv-CD19 proteins by flow cytometry analyses using ex vivo HIV-1-eGFP-infected CD4+ T cells or B cells, respectively, from YF-17D vaccinated study participants.

FIG. 3 shows induction of potent response of yellow fever-specific CD8+ T cells by YF-17D vaccination. (A) YF-17D vaccination pilot study showing frequency of YF epitope-specific CD8+ T cells in total circulating CD8+ T cells from YF-17D vaccinated individuals at baseline prior to vaccination and at day 17, 28, 36 and 95 post vaccination (n=2, data shown as mean+/−SEM). (B) The frequency of tetramer+ CD8+ T cells in total CD8+ T cells from 47 vaccinated study participants obtained at day 21+/−3 post vaccination. Data is plotted according to the HLA-type of the donors. Data for each individual donor is depicted with circles, and filled data points denotes values for PBMCs used in in vitro cell killing assays. The box plot shows the median and quartile range while the whiskers show minimum and maximum values for each HLA-type. (C) Phenotypic characterization of YF epitope-specific CD8+ T cells from 47 YF-17D vaccinated study participants based on surface staining for CD45RA and CCR7 into the subsets: Naive (CD45RA+, CCR7+), central memory (CM) (CD45RA−, CCR7+), effector memory (EM) (CD45RA−, CCR7−), and terminal differentiated (TD) (CD45RA+, CCR7−). Each bar represents the average subset composition of tetramer+ CD8+ T cells in individuals with the HLA-type denoted below the bar. The number of individuals in each analysis is denoted above the bars.

FIG. 4 shows specific killing of primary B target cells by autologous CD8+ T cells mediated by the proteinaceous compound. (A) Illustration of the workflow for in vitro target cell killing assays using the proteinaceous compound technology. (B-C) Killing assays of target B cells by CD8+ T cells from healthy donor PBMCs using the FDA-approved BiTE, Blincyto (n=3) for comparisons with in vitro cell killing assays using primary B cells and autologous CD8+ T cells obtained from YF-17D vaccinated study participants investigating the proteinaceous compound technology. More specifically, proteinaceous compound-mediated target cell killing was investigated in terms of specificity for the proteinaceous compound protein complex (n=5). All data shows the percentage of dead (Live/Dead stain) target cells (Celltrace+) out of total target cells and is shown as mean+/−SEM for all donors at different E:T or YF-E:T ratios (bar graph) or for individual donors at E:T or YF-E:T 1:1 (XY-graph). (+) including proteinaceous compound; (−) without proteinaceous compound. E: effector cells; T: target cells.

FIG. 5 shows the dependence on YF epitope-specific CD8+ T cells in in vitro target cell killing assays using CD8+ T cells obtained prior to or 21+/−3 days post YF-17D vaccination (bar graph: n=3, XY-graph: n=7). All data shows the percentage of dead (Live/Dead stain) target cells (Celltrace+) out of total target cells and is shown as mean+/−SEM for all donors at different E:T or YF-E:T ratios (bar graph) or for individual donors at E:T or YF-E:T 1:1 (XY-graph). (+) including proteinaceous compound; (−) without proteinaceous compound. E: effector cells; T: target cells.

FIG. 6 shows different proteinaceous compound HLA-I domains and YF epitopes together with autologous cells obtained from YF-17D vaccinated donors with matching HLA-type investigated in in vitro target B cell killing assays. (A-B) using a proteinaceous compound (CD19_A2) engaging CD19+B target cells and NS4B-HLA-A*02:01-specific CD8+ T effector cells (n=3) (A), or proteinaceous compound (CD19_B35) complex containing a NS2B-HLA-B*35:01 domain (n=2)(B). (+) including proteinaceous compound; (−) without proteinaceous compound. (C) HLA-specificity of proteinaceous compound-mediated killing was tested in in vitro cell killing assays using proteinaceous compounds containing HLA-I domains matching (HLApos) or different from (HLAneg) the HLA-type of the donor (n=3). All data shows the percentage of dead (Live/Dead stain) target cells (Celltrace+) out of total target cells and is shown as mean+/−SEM for all donors at different E:T or YF-E:T ratios (bar graph) or for individual donors at E:T or YF-E:T 1:1 (XY-graph). E: effector cells; T: target cells.

FIG. 7 shows comparison of cytokine release from killing assays using BiTE or a proteinaceous compound. Mesoscale multiplex analyses of supernatants from in vitro cell killing assays using Blincyto (n=2) or the proteinaceous compound with CD8+ T obtained from YF-17D vaccinated study participants prior to (n=3) or 21+/−3 days post vaccination (n=4). The analyzed cytokines released from the killing assays include IFN-γ (A-C), and TNF-α (D-F). All data is shown as mean+/−SEM. E: effector cells; T: target cells. ULOD: Upper Limit of Detection.

FIG. 8 shows specific killing of HIV-1 Env expressing target cells using the proteinaceous compound technology. (A) Neutralization data for scFv-10-1074 and scFv-CD19 showing specific binding of scFv-10-1074 to HIV-1 envelope (n=3). (B) Killing assay data of Raji-Env cells expressing HIV-1 Env and GFP using the proteinaceous compound technology (n=3). (C) Proteinaceous compound-mediated killing of autologous CD4+ T cells obtained from YF-17D vaccinated study participants and ex vivo infected with HIV-1-eGFP (n=3). All killing assay data shows the disappearance of HIV-1 Env-expressing target cells (GFP+) normalized to the number of target cells in control wells without exposure to proteinaceous compound. The killing data is shown as mean+/−SEM for all donors at different E:T or YF-E:T ratios (bar graph) or for individual donors at E:T or YF-E:T 1:1 (XY-graph). E: effector cells; T: target cells.

FIG. 9 shows specific killing of CD4 expressing target cells using the proteinaceous compound technology using autologous CD4+ T cells obtained from YF-17D vaccinated study participants. The killing data is shown as mean+/−SEM for all donors at different E:T or YF-E:T ratios (bar graph) or for individual donors at E:T or YF-E:T 1:1 (XYgraph). E: effector cells; T: target cells.

FIG. 10 shows an embodiment of an optimised proteinaceous compound using the Knob-into-Hole technology. (A) Knob part of proteinaceous compound, (B) Hole part of proteinaceous compound, (C) Proteinaceous compound combined Knob part and Hole part. (D) Functional assessment of the capacity of tetramerized Knob-into-Hole format proteinaceous compounds containing a HLA-A*02:01 domain with a YF epitope to engage YF-specific effector cells by flow cytometry analyses using CD8+ T cells from a YF-17D vaccinated HLA-A*02:01-positive study participant or a non-vaccinated HLA-A*02:01-negative study participant.

The present invention will now be described in more detail in the following.

### DETAILED DESCRIPTION OF THE INVENTION

### Definitions

Prior to discussing the present invention in further details, the following terms and conventions will first be defined:

### Proteinaceous Compound

In the present context, the term “proteinaceous compound” is to be understood as a compound mainly composed of amino acids forming a peptide or protein. The proteinaceous compound may be formed by one or more independent subunits such as peptides, which may be covalently or non-covalently linked.

In a specific embodiment, the proteinaceous compound is formed by two subunits linked via non-covalent coupling.

### Non-Covalent Coupling

In the present context, the term “non-covalent coupling” means any bonding via other interactions than a covalent bond. A non-covalent bond may be formed by e.g. hydrophobic interactions, hydrophilic interactions, ionic interactions, van der walls forces, hydrogen bonding, and combinations thereof.

### First Binding Moiety/Second Binding Moiety

In the present context, the term “binding moiety” such as first binding moiety or second binding moiety means the part of the protinaceous compound being able to bind to the target of interest.

The binding moiety may be an antibody or a fragment thereof.

### Target of Interest

In the present context, the term “target of interest” refers to a protein and/or glycostructures expressed on the surface of the target cell, and which the binding moiety of the proteinaceous compound has affinity for.

### Cell Surface Marker

In the present context, the term “cell surface marker” refers to a ligand such as a protein expressed on the surface of a cell. A cell surface marker may be a target of interest.

### Target Cell

In the present context, the term “target cell” refers to a cell expressing the target of interest e.g. on the surface of the cell, to be recognised by the binding moiety. Further, the target cell is to be destroyed by the CD8+ T cell. Examples of target cells are cancerous cells and cells infected by an intracellular pathogen e.g. a virus.

### Mammal

In the present context, the term “mammal” refers to a human, a racing animal, a lifestock or a companion animal. Most relevant mammals are humans, racing animals such as horses and camels, companion animals such as cats and dogs, lifestock such a pig, cattle, sheep.

Thus, in an embodiment, the subject is a mammal.

### CD8+ T-Cell

In the present context, the term “CD8+ T-cell” also refers to “a cytotoxic T cell” (also known as “TC”, “cytotoxic T lymphocyte”, “CTL”, “T-killer cell”, “cytolytic T cell”, “CD8+ T-cell” or “killer T cell”), is a T lymphocyte (a type of white blood cell) that kills cancer cells, cells that are infected (particularly with viruses), or cells that are damaged in other ways.

CD8+ T-cells are also described as effector cells according to the present invention.

### Antigenic Peptide

In the present context, the term “antigenic peptide” refers to a peptide that is capable of binding to a major histocompatibility complex (MHC) molecule to form a pMHC. The MHC presents the antigenic peptide to immune cells to induce a T-cell receptor dependent immune response.

### Epitope

In the present context, the term “epitope” means the antigenic determinant recognized by the TCR of the T cell and/or by the binding moiety.

### MHC

In the present context, the term “MHC” or “MHC molecule” refers to the major histocompatibility complex, a protein complex whose main function is to bind antigenic peptides derived from pathogens and display them on the cell surface for recognition by the appropriate T-cells.

There are two major classes of MHC molecules, MHC class I molecules and MHC class II molecules. Herein, “MHC” refers to MHC class I molecules. MHC class I molecules consists of an alpha-chain (heavy chain) produced by MHC genes and a beta-chain (light chain or β2-microglubulin [B2M]) produced by the β2-microglubulin gene.

The heavy chain consists of three domains denoted alpha-1, alpha-2 and alpha-3, respectively. The alpha-1 domain is located next to the non-covalently associated β2-microglubulin. The alpha-3 domain is a transmembrane domain, which anchors the MHC class I molecule in the cell membrane. Together, the alpha-1 and alpha-2 domains forms a heterodimer containing a peptide-binding groove, which bind a specific antigenic peptide. The amino acid sequence of the peptide-binding groove is the determinant as to which specific antigenic peptide is bound to the MHC class I molecule.

The MHC molecule may be a MHC class I molecule.

A pMHC refers to an MHC molecule with bound antigenic peptide.

### Human Leukocyte Antigen (HLA)

In humans, the MHC molecule is encoded by the human leukocyte antigen (HLA) gene complex. Thus, in the present context, the term “MHC” encompasses also “HLA”. There exist three major types of HLA and therefore MHC in the present context include, but are not limited to, HLA alleles that are coded in the gene loci for HLA-A, HLA-B, and HLA-C. Similarly, MHC include, but are not limited to, MHC class I-like molecules such as HLA-E, HLA-F, HLA-G, HLA-H, MIC A, MIC B, CD1d, ULBP-1, ULBP-2, and ULBP-3.

The human leukocyte antigen (HLA) system or complex is a complex of genes on chromosome 6 in humans, which encode cell-surface proteins responsible for the regulation of the immune system. The HLA system is also known as the human version of the major histocompatibility complex (MHC) found in many animals.

pMHC

In the present context, the term “pMHC” refers to a MHC molecule as defined above to which is bound an antigenic peptide. Thus, the term pMHC refers to MHC class I molecules loaded with antigenic peptide.

### Pharmaceutical Composition

In the present context, the term “pharmaceutical composition” refers to a composition comprising one or more proteinaceous compounds according to the invention, suspended in a suitable amount of a pharmaceutical acceptable diluent or excipient and/or a pharmaceutically acceptable carrier.

### Pharmaceutically Acceptable

In the present context, the term “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans.

### Excipient

In the present context, the term “excipient” refers to a diluent, adjuvant, carrier, or vehicle with which the composition of the invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

### Sequence Identity

In the present context, the term “sequence identity” is here defined as the sequence identity between genes or proteins at the nucleotide, base or amino acid level, respectively. Specifically, a DNA and a RNA sequence are considered identical if the transcript of the DNA sequence can be transcribed to the identical RNA sequence.

Thus, in the present context “sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned.

To determine the percent identity of two amino acid sequences or of two nucleic acids, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment, the two sequences are the same length.

In another embodiment, the two sequences are of different length and gaps are seen as different positions. One may manually align the sequences and count the number of identical amino acids. Alternatively, alignment of two sequences for the determination of percent identity may be accomplished using a mathematical algorithm. Such an algorithm is incorporated into the NBLAST and XBLAST programs of (Altschul et al. 1990). BLAST nucleotide searches may be performed with the NBLAST program, score=100, wordlength=12, to obtain nucleotide sequences homologous to a nucleic acid molecules of the invention. BLAST protein searches may be performed with the XBLAST program, score=50, wordlength=3 to obtain amino acid sequences homologous to a protein molecule of the invention.

To obtain gapped alignments for comparison purposes, Gapped BLAST may be utilized. Alternatively, PSI-Blast may be used to perform an iterated search, which detects distant relationships between molecules. When utilizing the NBLAST, XBLAST, and Gapped BLAST programs, the default parameters of the respective programs may be used. See http://www.ncbi.nlm.nih.gov. Alternatively, sequence identity may be calculated after the sequences have been aligned e.g. by the BLAST program in the EMBL database (www.ncbi.nlm.gov/cgi-bin/BLAST). Generally, the default settings with respect to e.g. “scoring matrix” and “gap penalty” may be used for alignment. In the context of the present invention, the BLASTN and PSI BLAST default settings may be advantageous.

The percent identity between two sequences may be determined using techniques similar to those described above, with or without allowing gaps. In calculating percent identity, only exact matches are counted. An embodiment of the present invention thus relates to sequences of the present invention that has some degree of sequence variation.

### Cancer

In the present context, the term “cancer” is to be interpreted as all types of diseases diagnosed as cancers including both solid tumors and haematological cancers.

### Intracellular Pathogen

In the present context, the term “intracellular pathogen” is to be interpreted as all types of pathogens capable of growing and reproducing inside a cell of a host. In particular, the pathogen may be a virus, like immunodeficiency virus (HIV), hepatitis virus or human T-cell lymphotropic virus type 1 (HTLV).

### Vaccination

In the present context, the term “vaccination” is the administration of a vaccine to a subject to produce immunity against a disease. The vaccine may be attenuated pathogens, dead pathogens, a protein or part of a protein (from the pathogen) known to exert an antigenic effect, a nucleic acid encoding a protein or part of a protein (from the pathogen) or similar.

### Proteinaceous Compound

As mentioned above, the herein disclosed proteinaceous compound is able to bring effective CD8+ T-cells in close proximity to target cells. Hereby, the target cells may be selectively killed by the CD8+ T cells as demonstrated by examples 5-10. The proteinaceous compound can be adapted to different targets of interested such as CD19 (example 5), HIV-1 envelope (example 9) and CD4 (example 10). Furthermore, the proteinaceous compound can be adapted to various pMHCs (example 7). Thus, in one aspect, the present invention relates to a proteinaceous compound comprising:
a) a first binding moiety, such as an antibody or fragment thereof, having affinity for a target of interest, and
b) a pMHC comprising an MHC molecule, such as an HLA molecule, presenting an antigenic peptide, wherein the presented antigenic peptide has affinity for CD8+ T-cells induced by the antigenic peptide, such as induced by vaccination.

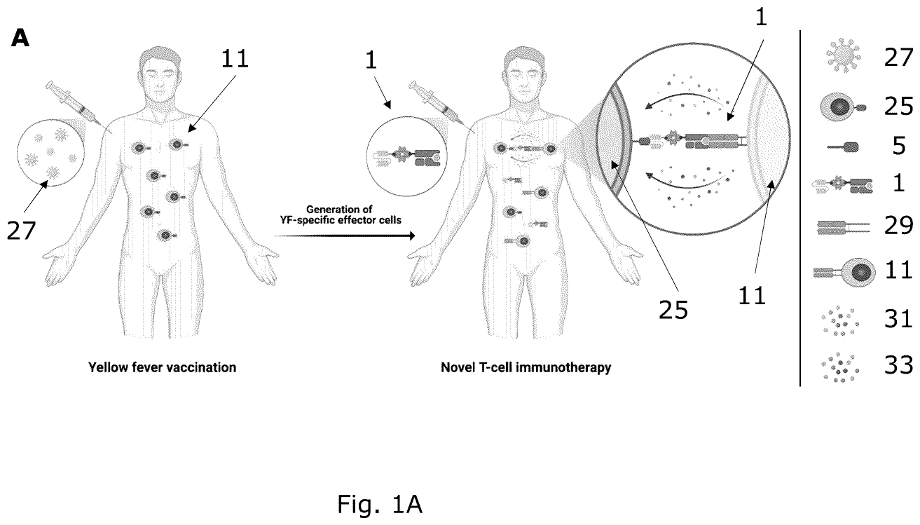

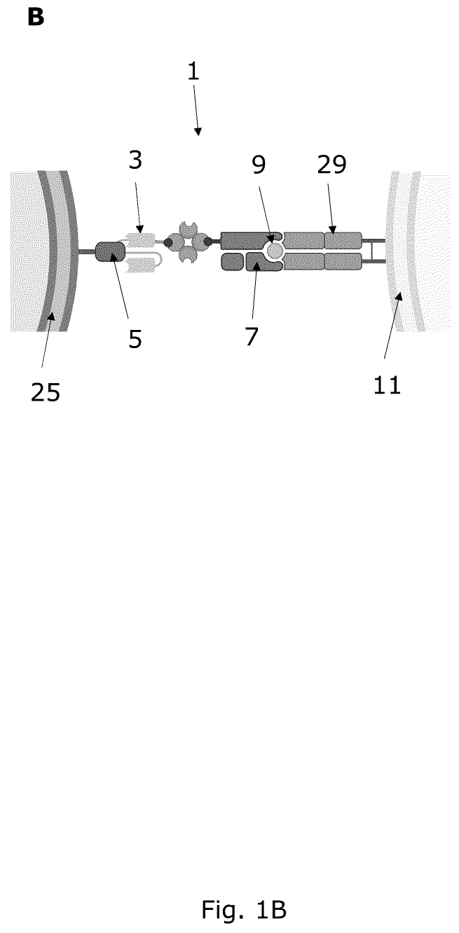

The first binding moiety has affinity for a target of interest and is able to connect the proteinaceous compound to the target cell via a target of interest. The pMHC comprises or consists of an MHC molecule presenting an antigenic peptide. This antigenic peptide has affinity for and is able to connect to the TCR of the CD8+ T-cells. Hereby, the target cell and the CD8+ T-cells are brought into proximity of one another enabling the killing of the target cell. Thus, in one embodiment, in vivo administration of the compound to a subject previously immunized with the antigenic peptide, such as by vaccination, will bring an induced CD8+ T-cell in proximity to cells presenting the target of interest, via the proteinaceous compound. Hence, the compound may function as a “linker” between a target interest on the target cell and CD8+ T-cells induced against the antigenic peptide. The principle of the present invention is illustrated in FIG. 1.

To be able to achieve this effect, it is essential that the TCR of the CD8+ T-cells is able to bind to the proteinaceous compound. Thus, the CD8+ T-cells needs to be induced by a comparable antigenic peptide. Hence, it is to be understood that the antigenic peptide in the proteinaceous compound should be identical to the antigenic peptide used e.g. in a vaccination process or at least should be sufficiently similar e.g. comprising a sufficiently similar epitope to enable to recognition of the induced CD8+ T-cells for the proteinaceous compound. Accordingly, the proteinaceous compound is suitable for in vivo administration to a subject previously immunized against the antigenic peptide, such as by vaccination. In one embodiment, the antigenic peptide of the proteinaceous compound and the antigenic peptide used in the vaccination are identical.

The size of the proteinaceous compound would be dependent upon the size of the pMHC as well as the binding moiety but also to potential other components of the proteinaceous compound such as amino acid linkers providing flexibility and linkers providing dimerization ability such as human immunoglobulin constant regions. All of these may be included in the proteinaceous compound to obtain a proteinaceous compound to be functional for the particular purpose.

In one embodiment, the proteinaceous compound has a molecular weight in the range of 30-160 kD, such as 40-140 kD, like 50-120 kD, such as 60-100 kD, preferably 65-95 kD.

In a further embodiment, the proteinaceous compound has a length in the range 250-1500 amino acids, such as 300-1300 amino acids, like 400-1100 amino acids, such as 500-950 amino acids, preferably 600-850 amino acids.

The design of the proteinaceous compound may be modified depending on the linkage between the first binding moiety and the pMHC. Hence, in one embodiment, the first binding moiety and the pMHC are covalently or non-covalently linked.

In order to create flexibility and be able for the first binding moiety as well as the pMHC to fold properly, they may be separated via a linker. Hence, in a further embodiment, the pMHC and the first binding moiety are covalently linked via a linker. The linker may be selected from the group of SEQ ID NO: 1, SEQ ID NO: 2 and SEQ ID NO: 3. Alternatively, the linker may be an Fc part of an antibody or other linkers known to the person skilled in the art.

Alternatively, the first binding moiety and the pMHC may be prepared separately and following connected by a coupling system such as a streptavidin-biotin system. In a further embodiment, the first and the pMHC are linked via a streptavidin-biotin system. In a still further embodiment, the first binding moiety is biotinylated and the pMHC is biotin-conjugated.

### Binding Moiety and Target of Interest

The binding moiety of the proteinaceous compound is decisive on the target of interest to be recognised. The proteinaceous compound comprises a first binding moiety but for some diseases the chosen target of interest may be downregulated for the target cell to escape killing by the immune system. Therefore, it may be advantageous in some embodiments for the proteinaceous compound to be able to detect more than one target of interest to avoid e.g. cancer resistance. Thus, in one embodiment, the proteinaceous compound comprises a second binding moiety, such as an antibody or fragment thereof, having affinity for a target of interest.

In a further embodiment, the first binding moiety and second binding moiety have affinity for different targets of interest, such as different targets of interest in relation to the same disease. In a further embodiment, the first binding moiety and second binding moiety have affinity for different targets, such as two different cancer cell surface targets or two different epitopes for the same virus, such as two different HIV epitopes.

Alternatively, the first and second binding moieties may be present on the proteinaceous compound, which bind to the same target of interest. This could either be in order to obtain a stronger binding to the target cell by binding to more targets of interest on the same cell. Then again, the binding moiety could bind to different cells having the same target of interest. In a further embodiment, the first binding moiety and second binding moiety have affinity for the same target of interest.

It is to be understood that the proteinaceous compound may comprise even further binding moieties than two such as a third and/or fourth binding moiety.

It is also to be understood that the coupling between the second or further binding moieties to the pMHC may be similar to the coupling between the first binding moiety and the pMHC.

The binding moiety may be any moiety capable of interacting with the target of interest in order to secure that the proteinaceous compound may bring the CD8+ T-cells into proximity of the target cell. In one embodiment, the first binding moiety and/or the second binding moiety is selected from the group consisting of polyclonal antibody, a monoclonal antibody, an antibody wherein the heavy chain and the light chain are connected by a flexible linker, an Fv molecule, an antigen binding fragment, a Fab fragment, a Fab′ fragment, a F(ab′)2 molecule, a fully human antibody, a humanized antibody, a chimeric antibody, scFv (single-chain variable fragment) and a single-domain antibody (sdAb) (nanobody or diabody), preferably a scFv (single-chain variable fragment) and a single-domain antibody (sdAb) (nanobody or diabody).

The target of interest could be any molecule. However, as the principle of the invention is to bring the target cell into close proximity to the CD8+ cells for destruction of the target cell, the target cell should be a cell such as a cancer cell or a cell comprising an intracellular pathogen. Thus, in a further embodiment, the first and/or second binding moiety has affinity for a target of interest, has affinity for
an intracellular pathogen, such as a virus, such as immunodeficiency virus (HIV), viral hepatitis or human T-cell lymphotropic virus type 1 (HTLV); or
a cancer cell surface protein, such as CD19, CD4, CD20, GD2, PSMA, or Mesothelin.

It is noted that for cells comprising e.g. human T-cell lymphotropic virus type 1 (HTLV), these cells would often also develop into cancerous cells.

In an even further embodiment, the first and/or second binding moiety having affinity for a target of interest, has affinity for a virus selected from the group consisting of HIV, such as HIV-1 or HIV-2, like HIV-1, viral hepatitis, such as chronical viral hepatitis, such as hepatitis A, B, C, D, or F and human T-cell lymphotropic virus type 1 (HTLV).

In a still further embodiment, the first and/or second binding moiety having affinity for a target of interest, has affinity for a cancer cell surface protein selected from the group consisting of CD19, CD4, CD20, GD2, PSMA, and Mesothelin, such as CD19 and CD4. In an even further embodiment, the first and/or second binding moiety having affinity for a target of interest, has affinity for a cancer cell surface protein CD19. In an even further embodiment, the first and/or second binding moiety having affinity for a target of interest, has affinity for a cancer cell surface protein CD4.

In a further embodiment, the first and/or second binding moiety having affinity for a target of interest, has affinity for a target presented on the surface of cell, such as a pathogenically derived target or a cancer derived target.

It is to be understood that any of the herein mentioned features relating to the first and/or second binding moiety would also relate to potential further binding moieties, such as a third and/or fourth binding moiety.

### MHC Molecule, Presenting an Antigenic Peptide

In order for the proteinaceous compound to be recognised by the CD8+ T-cells, it needs to present a recognisable antigenic peptide. The antigenic peptide is presented by an MHC molecule. In one embodiment, the MHC molecule is selected from the group consisting of HLA haplotype: A*01, A*02, A*03, A*24, A*28, A*31, B*07, B*08, B*15, B*35, B*44, B*50, such as the group consisting of HLA haplotype A*01, A*02, A*24, B*07, B*15, B*35, like the group consisting of HLA haplotype A*01:01, A*02:01, A*03:01, A*24:02, B*07:02, B*15:01, B*35:01, B*35:01, B*44:02 and B*57:01, such as the group consisting of HLA haplotype A*01:01, B*35:01 and A*02:01.

The part of the MHC molecule present in the proteinaceous compound is preferably the soluble part of the MHC molecule. Thus, in a further embodiment, said MHC molecule is truncated at the transmembrane region.

The combination of HLA haplotypes differs between subjects why it is advantageous for the HLA haplotype of the proteinaceous compound to match at least one haplotype of the subject. Thus, in a further embodiment, the MHC molecule matches the HLA-haplotype of a subject to which the compound is intended to be administered to.

To obtain an efficient effect of the proteinaceous compound, an immunodominant response where a powerful effect is obtained is advantageous. These responses are often obtained by using live-attenuated viruses for vaccination. Some immune responses developed against viruses are extraordinarily effective and rapidly clear the infection. An example is vaccination with the yellow fever vaccine (YFV). This vaccine is extremely effective in preventing yellow fever disease. The vaccine comprises a live-attenuated vaccine strain (YF-17D). This vaccination generates both high titer antibodies and a very potent CD8+ T cell response usually peaking at around day 21 following vaccination. A single immunization confers life-long immunity against yellow fever. Thus, in a still further embodiment, the antigenic peptide is derived from a virus, such as Yellow Fever Virus (YFV), measles, rubella, varicella or smallpox. In an even further embodiment, the antigenic peptide comprises or consists of the epitope of a vaccine, such as a Yellow Fever Virus (YFV), measles, rubella, varicella or smallpox vaccine.

In humans, it has been shown that CD8+ T cells recognize HLA-restricted epitopes in the viral E, NS1, NS2B, and NS3 proteins albeit with a strong immunodominant epitope in NS4B. Moreover, the T cell response to the yellow fever vaccine is remarkably expansive. Hence, in a further embodiment, the antigenic peptide comprises or consists of an epitope selected from the group consisting of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 and SEQ ID NO: 15 or fragments thereof.

The presentation of the antigenic peptide would depend on the HLA molecule, why it is observed that pairs of HLA-haplotypes and antigenic peptides results in the most optimal effects. Examples of such combinations are shown in Table 1. Thus, in a further embodiment, the pMHC comprises a MHC molecule presenting an antigenic peptide selected from the group consisting of SEQ ID NO: 6-A*01:01, SEQ ID NO: 7-A*02:01, SEQ ID NO: 8-A*03:01, SEQ ID NO: 9-A*24:02, SEQ ID NO: 10-B*07:02, SEQ ID NO: 11-B*15:01, SEQ ID NO: 12-B*35:01, SEQ ID NO: 13-B*35:01, SEQ ID NO: 14-B*44:02 and SEQ ID NO: 15-B*57:01 or fragments thereof, preferably selected from the group consisting of SEQ ID NO: 6-A*01:01, SEQ ID NO: 7-A*02:01, and SEQ ID NO: 12-B*35:01 or fragments thereof.

### Two-Part System

The proteinaceous compound may comprise two subunits that may be joined to form a functional proteinaceous compound. This may be beneficial for production purposes of the proteinaceous compound due to the complexity of the pMHC and the binding moiety. Hence, in a further embodiment, the proteinaceous compound comprises a first part and a second part linked covalently or non-covalently, preferably non-covalently, such as via a dimerization domain, like a Knob-into-Hole system. The Knob-into-Hole technique have successfully been used for dimerization as demonstrated in example 11. However, other dimerization systems may also be used such as c-jun transcription factor derived dimerization domains or leucine zipper dimerization domains. In one embodiment, the dimerization is a heterodimerization.

Designing the proteinaceous compound as a two-subunit system may also be advantageous with respect to having a second binding moiety. As an example the proteinaceous compound may be designed in two subunits where
said first subunit comprises said antigenic peptide of the pMHC, a first part of the MHC molecule such as the β2-microglobulin and said first binding moiety; and
said second subunit comprises a second part of said MHC molecule such as the MHC molecule without the β2-microglobin and said second binding moiety.

In a further embodiment, said first subunit and said second subunit further comprise a dimerization domain for enabling formation of a proteinaceous compound.

One dimerization system is the Knob-into-Hole system. This technology is a well-validated heterodimerization technology within the field of antibodies and relies on introducing a mutation for a large amino acid in one heavy chain and a mutation for a small amino acid in another constant region heavy chain. In an even further embodiment, said proteinaceous compound comprises said Knob-into-Hole system, such as the first subunit of the compound comprising the knob part and the second subunit of the compound comprising the hole part. Alternatively, said first subunit may comprise the hole part and the second subunit may comprise the knob part.

As an example, it has been demonstrated that the Fc-part of an IgG1 molecule may be mutated to form a Knob-into-Hole system promoting heterodimerization (Serra et al, 2020). A similar Knob-into-Hole system based on selected mutation of the Fc-part of an IgG1 molecule may also be used in the present invention for coupling a first and a second subunit to form a proteinaceous compound. Thus, in a further embodiment,
said knob part of the Knob-into-Hole system is defined by SEQ ID NO: 4 or at least 70% identity, such as 80% identity, like 90% identity, such as 95% identity, like 98% identity, such as 99% identity, to SEQ ID NO: 4, such as with the proviso that the amino acids at position 124 and position 139 are K; and
said hole part is defined by SEQ ID NO: 5 or at least 70% identity, such as 80% identity, like 90% identity, such as 95% identity, like 98% identity, such as 99% identity, to SEQ ID NO: 5 such as with the proviso that the amino acid at position 124 is D and position 141 is E.

The Knob-into-Hole system is comprised for coupling of the first and second subunits. Thus, this system needs to be arranged in the subunits in a manner allowing proper folding and interaction of the binding moieties and the pMHC with the target cells and effector cells, respectively. Thus, in an even further embodiment, said Knob-in-hole system is arranged between the pMHC and said first or second binding moiety.

Examples of first and second subunits designed with a Knob-into-Hole system are:
SEQ ID NO: 22-24: Second subunits comprising HLA*A02 (truncated at transmembrane region) and ScFv 10-1074 beside a knob part being human IgG1
SEQ ID NO: 25-27: First subunits comprising an antigenic peptide from NS4B viral protein, mature β2-microglobulin and ScFv 10-1074 beside a hole part being human IgG1.

These first and second subunits are preferably combined as follows: SEQ ID NO: 22 with SEQ ID NO: 25, SEQ ID NO: 23 with SEQ ID NO: 26 and SEQ ID NO: 24 with SEQ ID NO: 27. However, the first and second subunits may be combined in different order.

The exemplified second subunits comprise the following elements, where the human IgG1 comprises a KK mutation and the variable linker differs between SEQ ID NOs: 22-24.
K_{light} leader sequence (amino acids: 1-22)—HLA*A02 truncated at the transmembrane region (amino acids: 23-306)—Serra Linker (amino acids: 307-319)—human IgG1 (CH2 and CH2) L351K/T366K (amino acids: 320-540)—Linker1 (amino acids: 530-538)—ScFv 10-1074 (amino acids: 539-793)—Linker (G4S) (amino acids (amino acids: 794-798)—10×HIS (amino acids: 799-808) for SEQ ID NO: 22.
K_{light} leader sequence (amino acids: 1-22)—HLA*A02 truncated at the transmembrane region (amino acids: 23-306)—Serra Linker (amino acids: 307-319)—human IgG1 (CH2 and CH2) L351K/T366K (amino acids: 320-540)—Linker2 (amino acids: 530-542)—ScFv 10-1074 (amino acids: 543-797)—Linker (G4S) (amino acids (amino acids: 798-802) —10×HIS (amino acids: 803-812) for SEQ ID NO: 23.
K_{light} leader sequence (amino acids: 1-22)—HLA*A02 truncated at the transmembrane region (amino acids: 23-306)—Serra Linker (amino acids: 307-319)—human IgG1 (CH2 and CH2) L351K/T366K (amino acids: 320-540)—Linker3 (amino acids: 530-541)—ScFv 10-1074 (amino acids: 542-796)—Linker (G4S) (amino acids (amino acids: 797-801)—10×HIS (amino acids: 802-811) for SEQ ID NO: 24.

The exemplified first subunits comprise the following elements, where the human IgG1 comprises DE mutations and the variable linker differs between SEQ ID NOs: 25-27:
K_{light} leader sequence (amino acids: 1-22)—NS4B peptide (amino acids: 23-31)—Linker (G4S)₃ (amino acids: 32-46)—mature B2M (amino acids: 47-119)—Serra Linker (amino acids: 120-132)—human IgG1 (CH2 and CH2) L351K/T366K (amino acids: 133-351)—Linker1 (amino acids: 352-360)—ScFv 10-1074 (amino acids: 361-615) for SEQ ID NO: 25.
K_{light} leader sequence (amino acids: 1-22)—NS4B peptide (amino acids: 23-31)—Linker (G4S)₃ (amino acids: 32-46)—mature B2M (amino acids: 47-119)—Serra Linker (amino acids: 120-132)—human IgG1 (CH2 and CH2) L351K/T366K (amino acids: 133-351)—Linker2 (amino acids: 352-364)—ScFv 10-1074 (amino acids: 365-619) for SEQ ID NO: 26.
K_{light} leader sequence (amino acids: 1-22)—NS4B peptide (amino acids: 23-31)—Linker (G4S)₃ (amino acids: 32-46)—mature B2M (amino acids: 47-119)—Serra Linker (amino acids: 120-132)—human IgG1 (CH2 and CH2) L351K/T366K (amino acids: 133-351)—Linker3 (amino acids: 352-363)—ScFv 10-1074 (amino acids: 364-618) for SEQ ID NO: 27.

### CD8+ T-Cells

CD8+ T-cells are also known herein as effector cells as they are the cells to exert an effect on the target cells by killing them. The CD8+ T-cells are to be induced to be effective. This is performed by e.g. vaccination. Thus, in one embodiment, the antigenic peptide has affinity for CD8+ T-cells induced with the antigenic peptide by vaccination.

In a further embodiment, the vaccine is selected from the group consisting of YF-17D, MMRII and ACAM2000, such as YF-17D.

### Specific Compounds

In one embodiment, the proteinaceous compound as described herein comprises
a) a first binding moiety, such as an antibody or fragment thereof, having affinity for a target of interest, wherein said target of interest is derived from HIV, and
b) a pMHC comprising an MHC molecule, such as an HLA molecule, presenting an antigenic peptide, wherein the presented antigenic peptide is derived from Yellow Fever virus.

In one embodiment, the proteinaceous compound as described herein comprises
a) a first binding moiety, such as an antibody or fragment thereof, having affinity for a target of interest, wherein said target of interest is derived from HIV, wherein said binding moiety comprises or consists of an amino acid sequence selected from:
   i. SEQ ID NO: 17, or
   ii. an amino acid sequence having at least 80% sequence identity, like 90% identity, such as 95% identity, like 98% identity, such as 99% identity, to the sequence in (i); and
b) a pMHC comprising an MHC molecule being HLA-A*01:01, presenting an antigenic peptide, wherein the presented antigenic peptide is derived from Yellow Fever virus and comprises or consists of an amino acid sequence according to SEQ ID NO: 6.

In one embodiment, the proteinaceous compound as described herein comprises
a) a first binding moiety, such as an antibody or fragment thereof, having affinity for a target of interest, wherein said target of interest is derived from HIV, wherein said binding moiety comprises or consists of an amino acid sequence selected from:
   i. SEQ ID NO: 17, or
   ii. an amino acid sequence having at least 80% sequence, like 90% identity, such as 95% identity, like 98% identity, such as 99% identity, identity to the sequence in (i); and
b) a pMHC comprising an MHC molecule being HLA-A*02:01, presenting an antigenic peptide, wherein the presented antigenic peptide is derived from Yellow Fever virus and comprises or consists of an amino acid sequence according to SEQ ID NO: 7.

In one embodiment, the proteinaceous compound as described herein comprises
a) a first binding moiety, such as an antibody or fragment thereof, having affinity for a target of interest, wherein said target of interest is derived from HIV, wherein said binding moiety comprises or consists of an amino acid sequence selected from:
   i. SEQ ID NO: 17, or
   ii. an amino acid sequence having at least 80% sequence, like 90% identity, such as 95% identity, like 98% identity, such as 99% identity, identity to the sequence in (i); and
b) a pMHC comprising an MHC molecule being HLA-B*35:01, presenting an antigenic peptide, wherein the presented antigenic peptide is derived from Yellow Fever virus and comprises or consists of an amino acid sequence according to SEQ ID NO: 12.

In another embodiment, the proteinaceous compound as described herein comprises
a) a first binding moiety, such as an antibody or fragment thereof, having affinity for a target of interest, wherein said target of interest is a cancer surface marker, and
b) a pMHC comprising an MHC molecule, such as an HLA molecule, presenting an antigenic peptide, wherein the presented antigenic peptide is derived from Yellow Fever virus.

In another embodiment, the proteinaceous compound as described herein comprises
a) a first binding moiety, such as an antibody or fragment thereof, having affinity for a target of interest, wherein said target of interest is a cancer surface marker, wherein said binding moiety comprises or consists of an amino acid sequence selected from;
   i. SEQ ID NO: 16, or
   ii. An amino acid sequence having at least 80% sequence identity, like 90% identity, such as 95% identity, like 98% identity, such as 99% identity, to the sequence in (i); and
b) a pMHC comprising an MHC molecule being HLA-A*01:01, presenting an antigenic peptide, wherein the presented antigenic peptide is derived from Yellow Fever virus and comprises or consists of an amino acid sequence according to SEQ ID NO: 6.

In another embodiment, the proteinaceous compound as described herein comprises
a) a first binding moiety, such as an antibody or fragment thereof, having affinity for a target of interest, wherein said target of interest is a cancer surface marker, wherein said binding moiety comprises or consists of an amino acid sequence selected from;
   i. SEQ ID NO: 16, or
   ii. an amino acid sequence having at least 80% sequence identity, like 90% identity, such as 95% identity, like 98% identity, such as 99% identity, to the sequence in (i); and
b) a pMHC comprising an MHC molecule being HLA-A*02:01, presenting an antigenic peptide, wherein the presented antigenic peptide is derived from Yellow Fever virus and comprises or consists of an amino acid sequence according to SEQ ID NO: 7.

In another embodiment, the proteinaceous compound as described herein comprises
a) a first binding moiety, such as an antibody or fragment thereof, having affinity for a target of interest, wherein said target of interest is a cancer surface marker, wherein said binding moiety comprises or consists of an amino acid sequence selected from;
   i. SEQ ID NO: 16, or
   ii. an amino acid sequence having at least 80% sequence identity, like 90% identity, such as 95% identity, like 98% identity, such as 99% identity, to the sequence in (i); and
b) a pMHC comprising an MHC molecule being HLA-B*35:01, presenting an antigenic peptide, wherein the presented antigenic peptide is derived from Yellow Fever virus and comprises or consists of an amino acid sequence according to SEQ ID NO: 12.

### Nucleic Acid

The proteinaceous compound according to the present invention may be encoded by one or more nucleic acids. Thus, a further aspect of the present invention relates to a nucleic acid encoding the proteinaceous compound as described herein, the first subunit of the proteinaceous compound as described herein and/or the second subunit of the proteinaceous compound as described herein.

### Vector

The nucleic acids according to the present invention may be expressed by one or more vectors. Thus, a further aspect of the present invention relates to a vector comprising at least one nucleic acid as described herein.

In one embodiment, the vector is in the form of a plasmid, such as an expression plasmid, circular single-stranded DNA or circular double-stranded DNA.

### Cell

The vectors may be expressing the nucleic acids in a cell. Thus, yet an aspect of the present invention relates to a cell comprising one or more vectors who alone or in combination encode the proteinaceous compound as described herein.

In one embodiment, the cell is a mammalian cell, such as HEK293, CHO and Vero.

### Methods of Production

The compounds of the invention may also be produced by common methods. Thus, in a further aspect, the invention relates to a method of producing the proteinaceous compound (1) of the disclosure, the method comprising:
a. providing a nucleic acid encoding the proteinaceous compound as described herein, or an expression vector comprising said nucleic acid;
b. introducing the nucleic acid or the vector into a cell, such as cells selected from the group consisting of: bacteria and eukaryotes;
c. growing the cell under conditions that allow for expression of the proteinaceous compound (1) from the nucleic acid or the vector;
d. Optionally, purifying the proteinaceous compound (1).

Further specific methods of how the compounds can be produced are described throughout examples 1-3. Optimized proteinaceous compounds can also be produced using the Knob-into-Hole technique as described in example 11.

### Pharmaceutical Composition

The proteinaceous compound according to the invention may be used as a pharmaceutical composition. Thus in a further aspect, the present invention relates to a pharmaceutical composition comprising the proteinaceous compound as described herein.

In one embodiment, the pharmaceutical composition comprises two or more different proteinaceous compounds, such as proteinaceous compounds having different MCH molecules and/or antigenic peptides. Hereby is to be understood that the pharmaceutical composition may comprise proteinaceaous compounds having different pMCH, different antigenic peptides and/or different binding moieties. As an example, the pharmaceutical composition may comprise proteinaceous compounds having three different pMCH molecules corresponding to three HLA haplotypes of the subject to be treated.

### Combination

The present invention relates to the principle of linking CD8+ T-cells induced by vaccination, to target cells to destruction hereof. Hence, a vaccine is to be used together with the proteinaceous compound. Thus, in a further aspect, the present invention relates to a combination comprising
the proteinaceous compound as described herein; and
a vaccine, wherein said vaccine is for inducing CD8+ T-cells directed against the antigenic peptide.

The vaccine for vaccination is preferably exhibiting an immunodominant response. In one embodiment, the vaccine of the combination is a Yellow Fever vaccine, such as YF-10D, with the proviso that the antigenic peptide is derived from Yellow Fever and being part of the vaccine epitope. In another embodiment, the vaccine of the combination is a Measles, Mumps and Rubella vaccine, such as MMRII, with the proviso that the antigenic peptide is derived from Measles, Mumps or Rubella and being part of the vaccine epitope. In a further embodiment, the vaccine of the combination is a Smallpox vaccine, such as ACAM2000, with the proviso that the antigenic peptide is derived from Smallpox and being part of the vaccine epitope.

The combination may be provided as a kit. Thus, in a still further embodiment, the combination is in the form of a kit comprising
a first container comprising the proteinaceous compound,
a second container comprising the vaccine; and
optionally, instructions for use.

It is to be understood that the proteinaceous compound in the composition may be in the form of a pharmaceutical composition. Thus, in a further embodiment, the proteinaceous compound is in a pharmaceutical composition.

### Medicament

The proteinaceous compound or the pharmaceutically composition may be used as a medicament. Thus, a further aspect according to the present invention relates to, the proteinaceous compound as described herein or the pharmaceutical composition as described herein for use as a medicament, in the treatment of a subject, wherein the subject has previously received a vaccine for inducing CD8+ T-cells directed against the antigenic peptide; and
wherein the first and/or second binding moiety, having affinity for a target of interest,
   has affinity for an epitope of the pathogen causing the disease, such as a virus; or
   has affinity for a cell surface marker of a cancer.

In one embodiment, said subject is a mammal.

In particular, the proteinaceous compound and/or pharmaceutical composition may be used for treating a subject infected with a pathogen. Immunotherapy against HIV infected cells would appear to promise a potential cure of HIV. Presently, HIV is treated with antiretroviral therapy (ART) leaving the viruses in a dormant stage but not curing the disease. Thus, in a further aspect of the present invention relates to the proteinaceous compound as described herein or the pharmaceutical composition as described herein for use in the treatment of a subject infected with a pathogen, such as HIV, wherein the subject has previously received a vaccine for inducing CD8+ T-cells directed against the antigenic peptide;
wherein the first and/or second binding moiety, having affinity for a target of interest, has affinity for an epitope of the pathogen causing the disease, such as a virus, such as HIV, like HIV-1 or HIV-2.

In a further embodiment, the first and/or second binding moiety, having affinity for a target of interest, has affinity for an epitope of HIV-1.

In particular, the proteinaceous compound and/or pharmaceutical composition may be used for treatment of a cancer. Cancer therapy is often multimodal involving small molecule chemotherapy, radiotherapy and surgical procedures. Recently, remarkable progress has been achieved in activating the immune system to fight cancerous cells. Thus, a still further aspect of the present invention relates to the proteinaceous compound as described herein or the pharmaceutical composition as described herein for use in the treatment of a cancer, wherein the subject has previously received a vaccine for inducing CD8+ T-cells directed against the antigenic peptide;
wherein the first and/or second binding moiety, having affinity for a target of interest, has affinity for a cell surface marker of the cancer.

In one embodiment, said cell surface marker is CD19, CD4, CD20, GD2, PSMA, or Mesothelin, such as CD19 or CD4. In a further embodiment, the first and/or second binding moiety having affinity for a target of interest, has affinity for a cancer cell surface protein CD19. In a further embodiment, the first and/or second binding moiety having affinity for a target of interest, has affinity for a cancer cell surface protein CD4.

In a further embodiment, said cancer is B cell lymphoma, acute lymphoblastic leukemia (ALL), chronic lymphocytic leukemia (CALL), Neuroblastoma, Prostate cancer, Ovarian cancer or Cervical cancer.

The functioning of the proteinaceous compound is depending on the induction of CD8+ T-cells by a vaccine, where the induction of the CD8+ T-cells will take place some days after vaccination. Thus, in a still further embodiment, the vaccine for inducing CD8+ T-cells directed against the antigenic peptide,
is administered at least 5 days, such as at least 10 days prior to administration of the proteinaceous compound or the pharmaceutical composition, preferably at least 10 days, or
is administered 5-100 days prior to administration of the proteinaceous compound or the pharmaceutical composition, such as 10-100 days, such as 10-50 days, or preferably such as 15-25 days prior to administration of the proteinaceous compound.

The combination may be used as a medicament. Thus, a further aspect of the present invention relates to the combination as described herein for use as a medicament.

In particular, the combination may be used for treating a subject infected with a pathogen. Thus, in a further aspect of the present invention relates to the combination as described herein for use in the treatment of a subject infected with a pathogen, such as HIV. In a further embodiment, the first and/or second binding moiety, having affinity for a target of interest, has affinity for an epitope of HIV-1.

In particular, the combination may be used for treatment of a cancer. Thus, a still further aspect of the present invention relates to the combination as described herein for use in the treatment of a cancer. In one embodiment, said cell surface marker is CD19, CD4, CD20, GD2, PSMA, or Mesothelin, such as CD19 or CD4. In a further embodiment, said cancer is B cell lymphoma, acute lymphoblastic leukemia (ALL), chronic lymphocytic leukemia (CLL), Neuroblastoma, Prostate cancer, Ovarian cancer or Cervical cancer.

The functioning of the combination is depending on the induction of CD8+ T-cells by the vaccine. Thus, in a still further embodiment, the vaccine,
is administered at least 5 days, such as at least 10 days prior to administration of the proteinaceous compound, preferably at least 10 days, or
is administered 5-100 days prior to administration of the proteinaceous compound, such as 10-100 days, such as 10-50 days, or preferably such as 15-25 days prior to administration of the proteinaceous compound.

The CD8+ T cells linked to the target cell via the proteinaceous compound are induced by a vaccine. Hence, a further aspect of the present invention relates to a vaccine suitable for inducing CD8+ T-cells directed against an antigenic peptide, for use in the treatment of a subject
infected with an intracellular pathogen, such as a virus, such as HIV; or
suffering from cancer,
   wherein the subject is scheduled for treatment with the proteinaceous compound as described herein or the pharmaceutical composition as described herein after treatment with the vaccine.

The cancers from which the subject is suffering may be cancers such as those herein described. Similarly, the intracellular pathogen infecting the subject may be any of the pathogens as herein described.

A further aspect of the present invention relates to a method of treating a subject infected with an intracellular pathogen, such as a virus, such as HIV; OR suffering from cancer, by
administering to said subject a vaccine for inducing CD8+ T-cells directed against an antigenic peptide; and
following administering to said subject a proteinaceous compound as described herein or a pharmaceutical composition as described herein
   wherein the first and/or second binding moiety, having affinity for a target of interest, has affinity for an epitope of the pathogen causing the disease, such as a virus; or has affinity for a cell surface marker of a cancer.

In one embodiment, said proteinaceous compound or said pharmaceutical composition is administered after
at least 5 days, such as at least 10 days, like at least 15 days, such as at least 20 days, or
5-100 days, such as 10-100 days, like 10-50 days, or preferably such as 15-25 days
   from the administration of said vaccine.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1—Overview of Technology

Cytotoxic T lymphocytes (CTLs) are potent killers of malignant and virus infected cells. However, in cancer patients and HIV-1-infected individuals, disease remission or clearance is often limited by an effector response displaying an exhausted phenotype and with limited capacity to control or eliminate the infected or malignant cells. This limitation of an inadequate effector response is overcome by the immunotherapy concept according to the present invention, where a potent de novo effector response is generated in vivo by simple vaccination and redirected towards target cells by a proteinaceous compound.

The principle of the proteinaceous compound 1 is demonstrated in FIG. 1A-B and described for an embodiment using Yellow Fever Virus vaccination. The principle would be similar for other types of vaccination. FIG. 1A shows a yellow fever vaccination in the form of attenuated yellow fever viruses 27 being administered to a subject for generation of yellow fever-specific effector cells 11 i.e. (CD8+ T-cells). After induction of the CD8+ T-cells having affinity for an antigenic peptide 9 from yellow fever virus, the proteinaceous compound 1 is administered to the subject. The proteinaceous compound 1 binds to the CD8+ T-cells 11 induced by the yellow fever vaccine via its antigenic peptide 9 and further binds to a target of interest 5 on a target cell 25 by means of a first binding moiety 3. Hereby, the effector cell 11 (CD8+ T-cells) and the target cell 25 are brought into close proximity, which enables killing of the target cell 25.

The proteinaceous compound 1 comprises two functionally distinct domains (FIG. 1B): 1) a first binding moiety 3 targeting specific targets of interest 5 such as cell surface antigens on target cells 25, and 2) a pMHC such as an HLA class I molecule 7 carrying an antigenic peptide 9 able to specifically engage with a T-cell receptor 29 and activate vaccine-induced effector T cells (CD8+ T-cells) 11.

By vaccinating patients with the YF vaccine to generate a cytotoxic T cell response and then administering the proteinaceous compound 1, this concept obviates the need for ex vivo genetic modification, expansion and transplantation of patient cells, as with the CAR T technology, by exploiting the patient's own immune response. More importantly, the side effects of activating the entire T cell compartment as seen with e.g. BiTEs may be prevented by this approach as it more specifically activates only the YF-specific CD8+ T cells 11 and not the entire CD3+ T cell population, thereby avoiding excessive immune activation and the development of cytokine release syndrome.

Besides being a safe, simple, and inexpensive means to generate vast numbers of effector CD8+ T cells 11 in vivo, another promise of this concept is that it may be amenable to any cell surface antigen. By altering the first binding moiety 3 of the proteinaceous compound 1, this immunotherapy concept is adaptable to any target of interest 5 and thus holds great potential for various diseases. Furthermore, this principle could be adaptable to other types of vaccinations by altering the pMHC.

### Example 2—Material and Methods

### Yf-17D Vaccinated Study Participants/Healthy Donors

Healthy adult participants were recruited in the study. A single dose of 0.5 ml 17D live-attenuated yellow fever vaccine strain was administered (Stamaril, Sanofi Pasteur). Individuals with a previous history of vaccination with YF-17D were self-reported. Blood samples were taken (up to 100 days) prior to and at two time points (day 21±3 and 100±40) after vaccination. The yellow fever vaccine recommendations were followed. Safety assessments done at every visit. Blood sampling were taken in EDTA tubes (BD Vacutainer® K2 EDTA), but a subgroup of participants had leukapheresis performed prior to and at day 21±3 after vaccination. Blood samples were processed within 4 hours of collection, and plasma samples were stored at −80° C. Peripheral blood mononuclear cells (PBMCs) were isolated by density gradient centrifugation. The absolute number of PBMCs was determined using an automated cell counter (CASY cell counter, Innovatis), and cells were cryopreserved in fetal bovine serum plus 10% DMSO.

### HLA-Typing of YF-Specific CD8+ T Cells

For all study participants, HLA class I (HLA-A, -B, -C) alleles were genotyped at the ASHI-accredited laboratory HistoGenetics (Ossining, NY) using sequencing-based typing (SBT).

### Quantification of YF-Specific CD8+ T Cells

YF epitope-specific CD8+ T cells in PBMCs isolated from YF-17D vaccinated study participants were quantified by tetramer staining and flow multicolor flow cytometry.

To detect various HLA types, ten different biotin-conjugated HLA class I molecules carrying immunogenic YF peptides (ImmunAware, #1001-03, 1002-09, 1016-04, 1020-04, 1048-05, 1058-02, 1072-02, 1088-02, 1105-02 or custom design) were analyzed (Table 1)

TABLE 1
Overview of HLA class I molecules carrying
immunogenic YF peptides

| Viral | Amino | Antigenic | SEQ | HLA |
|---|---|---|---|---|
| protein | acid no* | Peptide | ID NO | haplotype |
| NS5 | 286-295 | KSEYMTSWFY | 6 | A*01:01 |
| NS4B | 214-222 | LLWNGPMAV | 7 | A*02:01 |
| NS3 | 4-12 | VLWDIPTPK | 8 | A*03:01 |
| NS3 | 24-32 | IYGIFQSTF | 9 | A*24:02 |
| NS5 | 672-680 | RPIDDRFGL | 10 | B*07:02 |
| NS3 | 73-81 | SVKEDLVAY | 11 | B*15:01 |
| NS2A | 4-13 | HAVPFGLVSM | 12 | B*35:01 |
| NS2B | 110-118 | HPFALLLVL | 13 | B*35:01 |
| E | 200-209 | TESWIVDRQW | 14 | B*44:02 |
| NS1 | 116-124 | KTWGKNLVF | 15 | B*57:01 |

*The amino acid no. refers to the position in each viral protein. The complete genome of Yellow Fever Virus (GenBank: X03700.1).

The biotin-conjugated HLA class I monomers carrying an YF peptide were tetramerized using PE-conjugated streptavidin (BioLegend, #405204). Tetramerization was obtained by three sequential additions of biotinylated YF peptide-HLA class I monomers to Streptavidin-PE in a 4:1 molar ratio, each addition followed by an incubation step at 4° C. for 15 minutes according to the manufacturer's instructions. Aliquots of 1.5×10⁶ PBMCs/flow tube were incubated for 30 minutes in the dark at room temperature (RT) in 40 μL tetramer solution (according to the HLA type of the donor) prediluted in FACS buffer (PBS with 2% HI-FBS) to a final concentration of 30 nM. For each analyzed participant, one flow tube received no tetramer stain and thus served as an unstained control. Subsequently, the cells were washed in PBS and stained with 5 μL Live/Dead™ Fixable Near-IR Dead Cell Stain (Invitrogen, #L10119) prediluted 20× in PBS and incubated for 5 minutes at RT in the dark. The PBMCs were further analyzed for T cell markers (CD3, CD8), extracellular T cell activation markers (CD38, HLA-DR) and CCR7 and CD45RA expression to define CD8+ T cell subsets using a panel of fluorochrome-conjugated antibodies (BD Biosciences, #564001, 565192, 353230, 555488, 562444, 748339) in Brilliant Stain buffer (BD Biosciences, #563794) for 20 minutes at RT in the dark. Unstained controls without tetramer stain were incubated with fluorochrome-conjugated IgG1 kappa and IgG2a kappa isotype controls (BD Biosciences, #562438, 612765) instead of anti-CD38 and -HLA-DR antibodies, respectively. The cells were washed twice in FACS buffer and analyzed by flow cytometry (BD LSRFortessa™ X-20; BD Biosciences) using Diva software.

### Culture of Primary Cells and Cell Lines

Peripheral blood mononuclear cells (PBMCs) and primary B cells, CD4+ and CD8+ T cells obtained from YF-17D vaccinated study participants were cultured in RPMI-1640 medium (RPMI) (biowest, #L0498-500) supplemented with 10% Heat Inactivated Foetal Bovine Serum (HI-FBS) (SERANA, #S-FBS-SA-025), 1% Penicillin-Streptomycin (P/S) (biowest #L0022-100) and 1% IL-2 (Gibco, #PHC0023).

HEK 293T cells obtained from the ATCC (ATCC® CRL-3216™) were cultured in Dulbecco's Modified Eagle's Medium High Glucose (DMEM) (biowest, #L0102-500) supplemented with 10% HI-FBS and 1% P/S (cDMEM) at 37° C. in 5% CO₂ in a humified incubator.

Expi293-F (Gibco, A14527) and BirA-Expi293-F cells were cultured in Expi293 Expression Medium (Life Technologies, A1435101) in sterile Erlenmeyer culture flasks at 37° C. in 8% CO₂ in a humified incubator with shaking. BirA-Expi293-F cells were generated by transfection of Expi293-F cells with pDisplay-BirA-ER (Addgene, #20856) plasmid expressing BirA enzyme. To select for BirA-expressing cells, 0.5 mg/mL G418 selection (Sigma, #G8168) was added to the culture medium, and 0.2 μg/mL biotin (Sigma #B4639) was added as substrate for BirA enzyme activity for biotinylation of scFv proteins.

Raji-Env (Dufloo et al., 2019) cells with stable expression of YU2 envelope and GFP under puromycin selection were cultured at 37° C. in 5% CO₂ in RPMI supplemented with 10% HI-FBS and 1% P/S (cRPMI) and selected for transduced cells with the addition of 1 μg/mL puromycin (Gibco, #A11138-03) to the cell culture medium.

Primary CD4+ T cells infected with HIV-1-GFP were handled in a biosafety class 2+ laboratory (BSL2+) and cultured in cRPMI supplemented with 1% IL-2 (cRPMI+IL2) at 37° C. in 5% CO₂ in a humified incubator.

All cell lines were tested negative for mycoplasma.

### Isolation and Activation of Primary Cells

Primary CD4+ T cells, CD8+ T cells and B cells were isolated from human PBMCs by negative selection using the CD4+ T Cell Isolation Kit (Miltenyi Biotec, #130-096-533), CD8+ T Cell Isolation Kit (Miltenyi Biotec, #130-096-495), and the B Cell Isolation Kit II (Miltenyi Biotec, #130-091-151), respectively, according to the manufacturer's instructions. The isolated target cells were then cultured in cRPMI+IL-2 at 37° C. in 5% CO₂. CD4+ T cells isolated for HIV-1 infection were additionally activated by addition of 1 μg/mL Phytohemagglutinin (PHA, Remel, #R30852801) to the cell culture medium for 48 or 72 hours prior to infection.

Cloning of scFv

DNA sequences for scFv-CD19 or scFv-10-1074 were designed based on the CD19-targeting domain of the BiTE, blinatumomab, or the broadly neutralizing antibody, 10-1074, targeting HIV-1 envelope, respectively. The CD19-targeting domain having the amino acid sequence given by SEQ ID NO: 16 and the 10-1075 antibody the amino acid sequence given by SEQ ID NO: 17.

The scFv DNA sequences were ordered as gBlocks® Gene Fragments from Integrated DNA Technologies and comprised the following: Restriction site—Kozak sequence—Secretory signal peptide for immunoglobulin kappa light chain—Variable light fragment—3×(G₄S) linker—Variable heavy fragment—GS linker—AviTag—G₃S linker—His-tag—Restriction site.

For expression of the scFv proteins from mammalian cell lines, the scFv DNA sequences were cloned into a pcDNA3.1(+) backbone plasmid (Invitrogen, #V790-20) by restriction enzyme digestion using XbaI (Thermo Scientific, #ER0685) and BamHI (Thermo Scientific, #ER0051) or HindIII (Thermo Scientific, #ER0501) according to the manufacturer's instruction. The digested pcDNA3.1(+) backbone plasmid was phosphatase-treated using FastAP Thermosensitive Alkaline Phosphatase (Thermo Scientific, #EF0654) and ligated with the digested scFv insert DNA in a 1:3 molar ratio using T4 DNA ligase (Invitrogen, #15224-017) according to the manufacturer's instructions.

The cloned scFv-pcDNA3.1(+) plasmids (SEQ ID NO: 18-19; encoding the amino acid sequence according to SEQ ID NO: 29-30) were propagated under 50 μg/mL carbenicillin (Sigma, #C1389) selection by transformation into One Shot TOP10 Competent bacteria (Invitrogen, #C4040-03) and purified using NucleoBond® Xtra Midi kit (Macherey-Nagel, #740410.50) according to the manufacturer's instructions.

Subsequently, the plasmids were Sanger sequenced by Macrogen Europe using their universal primers, BGH-R and T7promoter, on each side of the pcDNA3.1(+) cloning site, and the sequences were analyzed using Geneious Prime 2019.1 software.

Production of Biotinylated scFv Proteins

Biotinylated scFv proteins were produced by transient transfection of HEK 293T or BirA-Expi293-F cells with expression plasmids encoding scFv-CD19 or scFv-10-1074 using PEI MAX® transfection reagent (Polysciences, #24765-1) in a 1:4 ratio of scFv-pcDNA3.1(+) DNA to PEI according to the manufacturer's instructions. Supernatants were collected on day 5 post transfection, and the scFv proteins were isolated on a HiTrap TALON crude column (Cytiva, #29048565) capturing His-tagged protein and eluted in PBS supplemented with 10% glycerol.

Western Blot of Biotinylated scFv Proteins

Protein samples were mixed in a 3:1 ratio with a reducing buffer solution consisting of 4× Laemmli Sample Buffer (Bio-Rad, #1610747) supplemented with 5% 1 M dithiothreitol (DTT) (Sigma, #43816) and heated at 95° C. for 5 minutes to denature the proteins. The denatured proteins were separated by SDS-PAGE on a Mini-PROTEAN® TGX Stain-Free™ Protein Gel 4-20% or a Criterion™ TGX Stain-Free™ Protein Gel 4-20% (Bio-Rad, #4568096/5678094/5678095) in 1× Tris/Glycine/SDS (TGS) buffer (Bio-Rad, #1610772) and transferred to a PVDF membrane (Bio-Rad, #1704156/ #1704157) using a Trans-Blot Turbo Transfer System (Biorad) according to the manufacturer's instructions. The membrane was blocked in Tris-buffered Saline (TBS) (Bio-Rad, #1706435) supplemented with 0.1% Tween®20 (Sigma, #β1379) (TBS-T) and 3% W/V Nonfat-dried Milk bovine powder (Sigma, M7409) for 1 hour at RT with rocking. Following blocking, the membrane was incubated with anti-Avi-tag (Genscript, #A00674) or horseradish peroxidase (HRP)-conjugated Direct-Blot™ anti-His tag (BioLegend, #362614) primary antibodies or streptavidin-HRP (Invitrogen™, S911) diluted 1:2000, 1:500 or 1:3333, respectively, in TBS-T buffer supplemented with 1% W/V Nonfat-dried Milk bovine powder overnight at 4° C. or at RT for 1 hour with rocking.

The membrane was washed 3 times in TBS-T for 10 minutes each with rocking. If applicable, the membrane was incubated with an HRP-conjugated anti-rabbit IgG (Genscript, #A01827) secondary antibody diluted 1:5000 in TBS-T supplemented with 1% Nonfat-dried Milk bovine powder for 1 hour at RT with rocking. The membrane was washed 3 times for 10 minutes each with rocking prior to visualization using Clarity™ Western ECL substrate (Biorad, #170-5060) and the ChemiDoc imaging system (Biorad) according to the manufacturer's instructions.

Functional Assessment scFvs

Primary CD4+ T cells infected with HIV-1-eGFP, primary B cells or Raji-Env cells were seeded in flow tubes at a density of 5.0×10⁵ cells/tube. Cells were washed in FACS buffer and incubated with 9.25 μg/mL of scFv-10-1074-biotin or 8.96 μg/mL scFv-CD19-biotin at 4° C. for 1 hour. After washing twice with FACS buffer, the cells were incubated with 10 μg/mL of BV421-conjugated Streptavidin (BioLegend, #405226) in the dark at 4° C. for 20 minutes. Finally, the cells were washed twice in FACS buffer (PBS+2% HI-FBS), fixed with 1% formaldehyde (Thermo Scientific, #28908), and analyzed by flow cytometry (MACSQuant® Analyzer 16).

### Preassembly of Proteinaceous Compound

The proteinaceous compound was preassembled according to the protocol for tetramerization of HLA-I monomers as described above, however replacing half the HLA-I molecules with scFv. Preassembly of proteinaceous compound was thus achieved by combining 1.627 mol scFv or HLA-I monomer to 1 mol Streptavidin. In in vitro cell killing assays, 1.8×10⁻⁵ μmol proteinaceous compound was used per 10⁶ target cells. In brief, the proteinaceous compound was preassembled by sequential additions of ⅓ of the HLA or scFv volume to streptavidin to ensure binding of both HLA and scFv. Moreover, a negative control of the proteinaceous compound lacking an HLA domain was assembled by substituting the addition of HLA with PBS. Specifically, ⅓ of the full biotin-conjugated HLA-I monomer volume or an equivalent volume of PBS was incubated with the full volume of Streptavidin at 4° C. for 15 minutes. Subsequently, ⅓ of the full scFv volume was added to the solution followed by incubation at 4° C. for 15 minutes. These steps were repeated twice to obtain binding of the full volume of HLA and scFv to the Streptavidin for assembly of the final proteinaceous compound.

### In Vitro Cell Killing Assays

To assess the capacity of the proteinaceous compound to mediate killing of target cells by vaccine-induced YF-specific CD8+ T cells and compare this to BiTE-mediated killing using Blincyto (blinatumomab, Amgen), in vitro cell killing assays using autologous cells obtained from CYF8 study participants were performed. Specifically, target B or CD4+ cells isolated from human PBMCs were stained with a 2.5 μM CellTrace™ Violet (Invitrogen, #C24557) solution for 20 minutes at 37° C. to be able to discriminate target cells i.e. cells comprising the target of interest from effector cells i.e. CD8+ T-cells directed against the antigenic peptide of the proteinaceous compound, by flow cytometry.

Subsequently, 9 mL cRPMI was added to the target cells and incubated at 37° C. for minutes in order to absorb unbound dye. The target cells were centrifuged at 300×g at RT for 8 minutes and resuspended in 100 μL FACS buffer (PBS+2% HI-FBS) for killing assays using proteinaceous compound or in cRPMI+IL2 to obtain a concentration of 5.0×10⁴ cells/mL for Blincyto-mediated killing assays. In killing assays using Blincyto, target cells were seeded in a round-bottom 96-well plate (Sarstedt, #83.3925.500) with 5.0×10³ cells/well. 50 μL cRPMI+IL2 or 10 ng/mL Blincyto was added to the target cells immediately prior to adding CD8+ effector cells in different effector to target (E:T) cell ratios in a total volume of 250 μL.

For proteinaceous compound-mediated killing assays, the target cells resuspended in FACS buffer solution were incubated with preassembled proteinaceous compound (see previous section) for 1 hour at 4° C. Following incubation, the target cells were washed in FACS buffer and resuspended in cRPMI+IL2 to a concentration of 5.0×10⁴ cells/mL. Target cells were seeded in a round-bottom 96-well plate (Sarstedt, #83.3925.500) at a density of 5.0×10³ cells/well, and autologous CD8+ T cells were added in different YF-specific effector to target (YF-E:T) cell ratios in a final volume of 200 μL. Each E:T was tested in triplicates.

After 20 hours incubation at 37° C. in 5% CO₂, the plate was centrifuged at 300×g for 8 minutes at RT, and 100 μL supernatant from each well was collected and saved at −80° C. for later cytokine analysis. To identify dead or dying cells, the cells in each well was stained with 2.5 μL of 10× diluted Live/Dead™ Fixable Near-IR Dead Cell Stain (LD-NIR) for 5 minutes in the dark at RT. The samples were then analyzed by flow cytometry for dead (LD-NIR+) target cells (CellTrace™+) using a MACSQuant® Analyzer 16.

### Cytokine Release Assays

The release of the cytokines IFN-γ and/or TNF-α in cell killing assays using the BiTE, Blincyto (blinatumomab), or the proteinaceous compound was quantified by mesoscale multiplex assays using V-PLEX Custom Human Biomarkers based on Proinflammatory Panel 1 Human Kit (Meso Scale Discovery, Inc., K151A9H-1) or U-PLEX Immuno-Oncology Group 1 (hu) (Meso Scale Discovery, Inc., K151AEL-1) according to the manufacturer's instructions. The samples were analyzed in duplicates on a MESO QuickPlex SQ 120 imager using DISCOVERY WORKBENCH® software.

### HIV-1-eGFP Virus Production

A full-length HIV-1-GFP reporter virus was generated using a pBR-HIV-1 M NL4-3 92TH14-12 plasmid (SEQ ID NO: 28). The pBR-HIV-1 M NL4-3 92TH14-12 plasmid encodes a full-length HIV-1 with a R5 tropic envelope. Additionally, a reporter gene sequence encoding enhanced Green Fluorescent Protein (eGFP) has been inserted into the HIV-1 genome immediately after nef gene.

The HIV-1-GFP viruses were generated by transfection of HEK 293T cells with the pBR-HIV-1 M NL4-3 92TH14-12 plasmid using PEI MAX® transfection reagent at a 4:1 molar ratio of PEI:DNA according to the manufacturer's instructions. Viral supernatants were collected 72 hours post transfection and filtered through a 0.45 μm Minisart® NML Syringe Filter (Sartorius, #16555-K). The viral supernatants were concentrated by centrifugation through an Amicon Ultra-15 100K centrifugal filter device (Millipore, #UFC910008) at 2500×g at 4° C. for 20 minutes and aliquoted prior to storing at −80° C. until use.

### HIV-1 Neutralization Assay

CD4+ T cells were seeded in cRPMI+IL2 at a density of 3.0×10⁴ cells/well in a round-bottom 96-well plate (Sarstedt, #83.3925.500) 48 hours post activation. Each scFv was 3-fold serially diluted in cRPMI+IL2 with a starting concentration of 25 μg/mL and incubated with HIV-1-eGFP at 37° C. for 1 hour. The medium was removed from the cells and 100 μL of scFv-virus mixture was transferred to the cells with a multiplicity of infection (MOI) of 0.1. Next, the plate was centrifuged at 1250×g at 30° C. for 2 hours followed by incubation at 37° C. in 5% CO₂. Each scFv dilution was tested in technical triplicates. After 24 hours incubation at 37° C., the cells were washed in FACS buffer, fixed with 1% formaldehyde, and analyzed by flow cytometry (MACSQuant® Analyzer 16) for GFP+ cells.

### HIV-1 Env-Expressing Target Cell Killing Assay

To assess proteinaceous compound-mediated killing of target cells expressing HIV-1 envelope, in vitro cell killing assays were performed using a human B lymphoblastoid Raji cell line stably expressing YU2 envelope and Green Fluorescent Protein (GFP) (Raji-Env), and autologous CD4+ T cells obtained from YF-17 vaccinated study participants ex vivo infected with HIV-1-eGFP. Specifically, autologous CD4+ T cells were isolated from human PBMCs and activated for 48 hours in cRPMI+IL2 supplemented with 1 μg/mL PHA at 37° C. in 5% CO₂.

Following activation, the PHA was removed from the cells by centrifugation at 300×g for 5 minutes at RT. The cells were resuspended in HIV-1-eGFP diluted in cRPMI+IL2 for a MOI of 0.05 and spinoculated at 1250×g for 2 hours at 30° C. Following spinoculation, the cells were incubated at 37° C. in 5% CO₂ overnight. The cells were diluted in Tyto buffer (Miltenyi, #130-107-207) to a concentration of 5×10⁵ cells/mL and sorted for GFP expression in a MACSQuant Tyto Cartridge (Miltenyi, #130-104-791) using a MACSQuant® Tyto® Cell Sorter (Miltenyi) in order to enrich the GFP+ target cell population. The HIV-1-eGFP-infected autologous CD4+ T cells or Raji-Env cells were seeded in triplicates in a round-bottom 96 well plate with 2000-4000 or 5000 GFP+ target cells/well, respectively, in an in vitro cell killing assay setup as described earlier. In this HIV-1 Env-expressing target cell killing assay, a preassembled Raji cell line with a scFv-10-1074 from the broadly neutralizing antibody 10-1074 targeting HIV-1 envelope was used. CD8+ T cells obtained from YF-17D vaccinated study participants were added in different YF-specific effector to GFP+ target (YF-E:T) cell ratios in a final volume of 200 μL and incubated at 37° C. in 5% CO₂ in a humified incubator. After 20 hours incubation, the plate was centrifuged at 300×g for 8 minutes at RT, and 100 μL supernatant was gently removed from each well. The HIV-1-eGFP-infected cells were fixated 1% formaldehyde and incubated for 15 minutes at RT. The samples were then analyzed by flow cytometry for GFP+ cells using a MACSQuant® Analyzer 16. Dead target cells were thus quantified by the disappearance of GFP+ cells.

### Statistics

Statistical analyses were performed using GraphPad Prism (version 9.2.0), and the statistical analysis methods used as well as the specifics of data presentation are reported in the figure legends. Paired data was analyzed using Wilcoxon test, whereas Mann-Whitney statistical test was used to analyze unpaired data. A p-value below 0.05 was considered statistically significant and denoted as *p<0.5, **p<0.01, ***p<0.001.

### Example 3—Functional scFvs

### Aim of Study

To produce and asses the function of scFv domains to be used in a proteinaceous compound.

### Material and Methods

See example 2

### Results

The proteinaceous compound is assembled from a biotinylated scFv protein and a biotin-conjugated HLA-I molecule carrying immunogenic YF epitopes using Streptavidin which have a strong binding capacity to biotinylated proteins.

The scFv sequence containing an AviTag and His-tag for biotinylation and purification purposes, respectively, were designed from the binding-specific Fab domain of antibodies targeting a specific antigen of choice. For assembly of proteinaceous compound to be used in in vitro cell killing assays with B cells and HIV-1 infected CD4+ T cells as targets, a scFv-CD19 and scFv-10-1074 have been designed on the basis of the CD19-targeting scFv domain of the BiTE, Blincyto, and the broadly neutralizing antibody, 10-1074, targeting HIV-1 envelope, respectively. For expression of the scFv proteins, the DNA constructs encoding these proteins were cloned into a pcDNA3.1(+) expression vectors by restriction enzyme digestion and ligation. The cloned plasmids have been transformed into chemically competent bacteria for propagation and have been verified by Sanger sequencing following purification.

To test the functional efficacy of the scFv domains to be used in the assembly of the proteinaceous compound, the scFv proteins have been produced by transfection of the expression plasmids into a mammalian suspension BirA-Expi293F cell line for largescale protein production and with expression of BirA ligase for biotinylation of AviTag inherent in the scFv sequence prior to secretion. Due to an inherent secretory signal in the sequences of the scFv, the proteins were secreted into the cell medium from where they were collected and purified using affinity chromatography columns capturing His-tagged proteins.

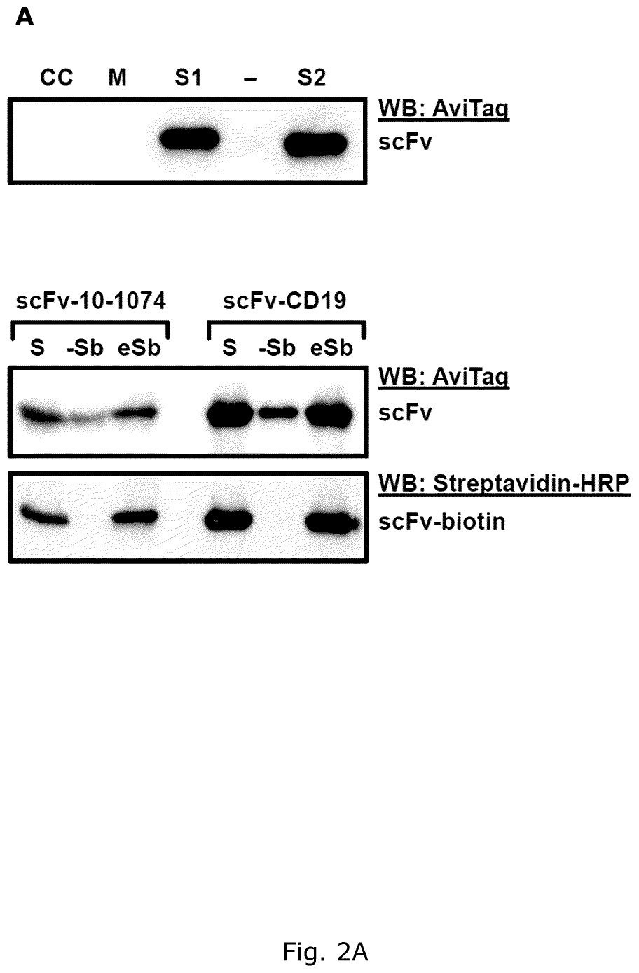

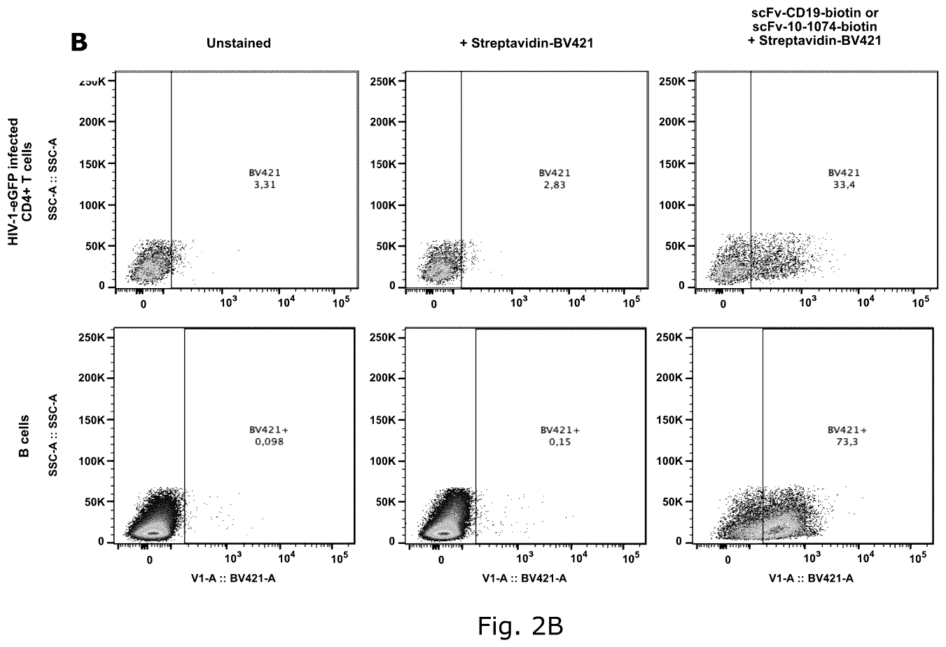

Production and successful biotinylation of the scFv proteins have been verified by western blot analysis (FIG. 2A) using an HRP-conjugated antibody or Streptavidin targeting the scFv AviTag (upper panel) or the biotinylated scFv proteins (lower panels), respectively. Densiometric analysis of the western blots visualized by HRP-conjugated Streptavidin showed that nearly 90% of scFv-10-1074 and scFv-CD19 were biotinylated, which is a prerequisite for successful assembly of a proteinaceous compound. Upper panel: cc=cell control, M=mock, S1=scFv-10-1074, S2=scFv-CD19. Lower panels: S=scFv, −Sb=scFv-biotin removed, eSb=eluted scFv-biotin.

Moreover, functional assessment of the scFv-10-1074 and scFv-CD19 proteins have been carried out by assays examining the binding capacity of the scFvs to target cells (FIG. 2B) using human CD19+ B cells and CD4+ T cells ex vivo infected with a HIV-1-eGFP reporter virus.

Tracking of scFv binding to the target cells was enabled by flow cytometry for GFP expression and/or a streptavidin-conjugated fluorochrome marking the biotinylated scFvs. As expected, the flow cytometry data for the scFv binding assay (FIG. 2B) showed a pronounced binding capacity of scFv-CD19 to CD19+ B cells of 73.3%, whereas 33.4% of scFv-10-1074 were shown to the HIV-1-eGFP-infected CD4+ target cells. This lower binding of scFv-10-1074 to HIV-1 infected CD4+ T cells compared to scFv-CD19 binding to B cells could be due to the dynamics of HIV-1 infection. CD19 is constitutively expressed in high numbers on the surface of B cells, whereas cell surface expression of HIV-1 envelope is transient, dependent on the stage of the HIV-1 replication cycle, and is only expressed in low numbers. This is also evident from assays examining scFv-10-1074 and scFv-CD19 binding capacity using a human B lymphoblastoid Raji-Env cell line (data not shown) with overexpression of GFP, CD19 and HIV-1 Env not dependent on a viral replication cycle. The flow cytometry data for the scFv binding assay with Raji-Env cells (data not shown) showed pronounced binding of the scFv-CD19 (94.0%) to the Raji-Env cell line, approaching the level of control anti-CD19 antibody binding (99.1%) to the cells. Additionally, 76% of scFv-10-1074 bound the HIV-1 Env expressing Raji-Env target cells, thereby validating scFv function.

### Conclusion

scFv domains targeting CD19 and HIV-1 envelope can be successfully produced and show to bind to the targets of interest on target cells.

### Example 4—Effect of Yellow Fever Vaccinations

### Aim of Study

To study the effect of Yellow fever vaccination on the generation of specific CD8+ T cells.

### Material and Methods

See example 2.

### Results

For killing of target cells, the proteinaceous compound technology takes advantage of the effector response induced by vaccination. The YF-17D vaccine is known as one of the most successful vaccines ever developed due to its high effectivity and safety. The immune response elicited by this vaccine is exceptionally strong and of broad specificity targeting several epitopes within the viral proteins, NS1, NS2A, NS2B, NS3, NS4A, NS4B, NS5, C, M, and E, with an immunodominant HLA-A2-restricted epitope in the NS4B protein. In addition, peak responses up to 10% of all circulating CD8+ T cells have shown to be specific for YF epitope, thus underlining the strength of the CD8+ T cell response generated by vaccination with the YF-17D vaccine.

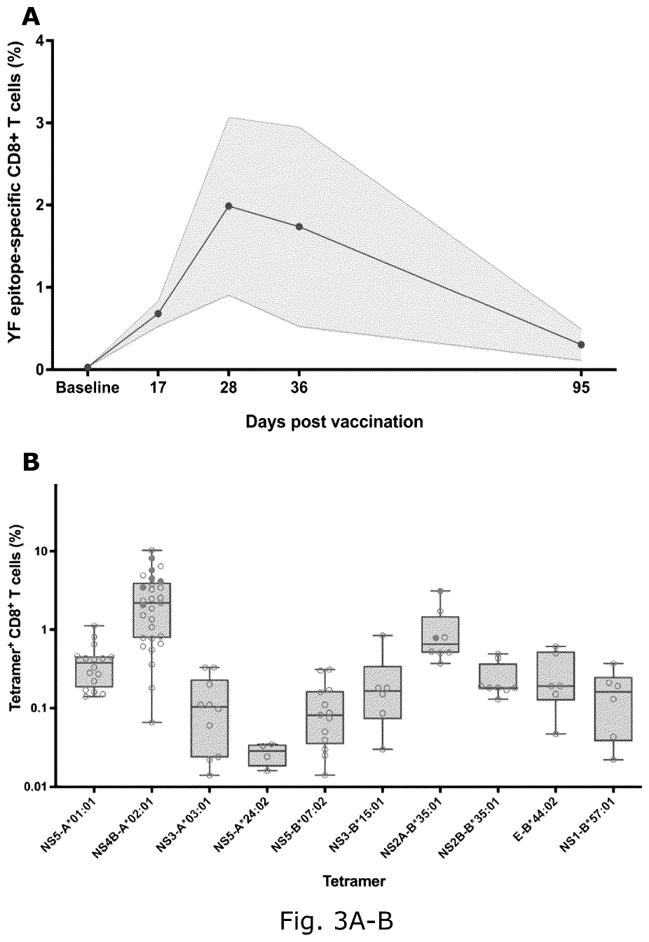

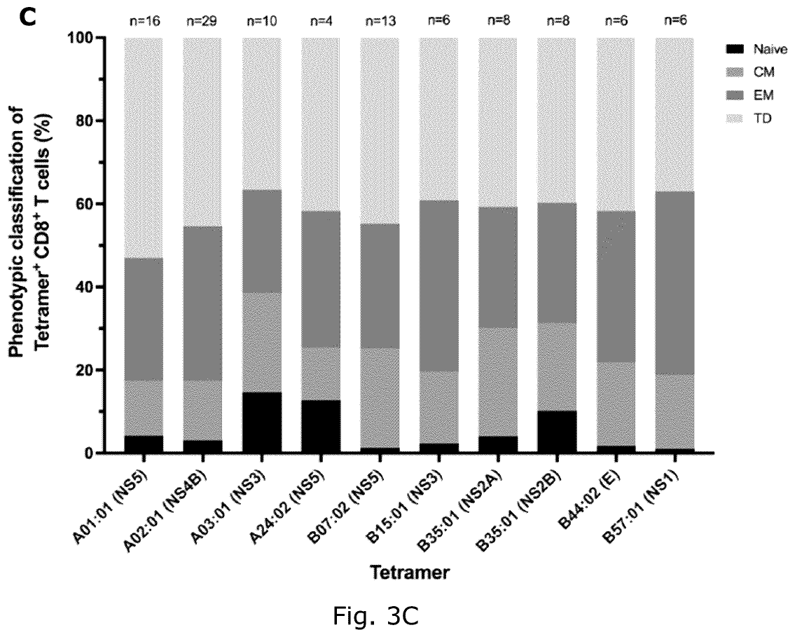

Vaccination of 51 healthy volunteers with the life attenuated YF-17D vaccine (Stamaril, Novartis) against yellow fever (YF) was performed to obtain YF epitope-specific CD8+ T cells and autologous target B and CD4+ T cells to be used in killing assays for assessment of the functional efficacy of proteinaceous compound constructs. As expected from the literature and shown by a pilot study of YF-17D vaccination of two healthy individuals (FIG. 3A), YF-17D vaccination generates a potent CD8+ T cell response that peaks between day 14 and 30 following vaccination. Thus, PBMCs from YF-17D-vaccinated study participants were collected at baseline (day−100 to 0 prior to vaccination), at peak response (day 21+/−3 days post vaccination) and at follow-up (day 100+/−40 days post vaccination) to enable comparison of the effect before and after vaccination.

To quantify YF-epitope specific CD8+ T cells in vaccinated study participants, PBMCs isolated at day 21+/−3 days following YF-17D vaccination were stained with a panel of fluorochrome-conjugated antibodies for T cell markers along with YF-peptide-HLA-I tetramers according to the HLA-type of the donor and analyzed by flow cytometry for tetramer+ CD8+ T cells. Amongst the YF-17D vaccinated study participants, 79% had YF-specific CD8+ T cells targeting more than one of the viral proteins or epitopes within these proteins assessed in this study. Furthermore, YF-specific CD8+ T cells were detected for all the included epitopes, demonstrating the broad specificity of the CD8+ T cell responses induced by the yellow fever vaccine. Generally, the magnitude of the responses to the different epitopes varied greatly among the analyzed donors regardless of epitope specificity. The greatest frequencies of YF-specific CD8+ T cells at day 21+/−3 days following YF-17D vaccination was found amongst donors within CD8+ T cells recognizing the HLA-A1, -A2 and -B35 restricted epitopes NS5, NS4B and NS2A, respectively. At this time point, NS5-HLA-A*01:01-specific CD8+ T cell responses ranged from 0.14% to 1.12% of the total pool of circulating CD8+ T cells, while NS2A-HLA-B*35:01-specific CD8+ T cells ranged from 0.37% to 3.13% of the total CD8+ T cells. The NS4B epitope in the context of HLA-A*02:01 led to the strongest response with up to 10.3% (range 0.07-10.30%) of all circulating CD8+ T cells in the blood being YF-specific (FIG. 3B).

Further characterization of the YF-specific CD8+ T cells in respect to phenotype was performed based on the expression of CD45RA and CCR7. Evaluation of the cells in respect to these two surface markers allows the YF-specific CD8+ T cell population to be divided into four subsets; naive (N) (CCR7+, CD45RA+), central memory (CM) (CCR7+, CD45RA−), effector memory (EM) (CCR7−, CD45RA−) and terminally differentiated (TD) (CCR7−, CD45RA+) T cells. Generally, the YF-specific CD8+ T cells showed a very similar phenotype distribution across CD8+ T cell specificities with the TD (CD45RA+, CCR7−) and the EM (CD45RA−, CCR7−) subsets, which are indicative of high cytotoxicity, comprising the majority of the YF-specific CD8+ T cells (FIG. 3C).

### Conclusion

In conclusion, at day 21+/−3 days following vaccination, YF-specific CD8+ T cells are predominantly of the TD and EM phenotypes regardless of epitope-specificity. Based on the general strong expansion of CD8+ T cells following vaccination in the included study participants carrying these HLA-I alleles and the known immunodominance of the HLA-A2 restricted epitopes in NS4B, the HLA-epitopes NS4B-HLA-A*02:01, NS5-HLA-A*01:01 and NS2A-HLA-B*35:01 were selected for use in the later killing assays. Thus, a variety of HLA types was included to explore the capacity of different HLA-epitopes to redirect YF-specific CD8+ T cells.

### Example 5—CD19 Target Killing

### Aim of Study

To investigate the killing effect of the proteinaceous compound on target cells expressing CD19.

### Material and Methods

See example 2.

### Results

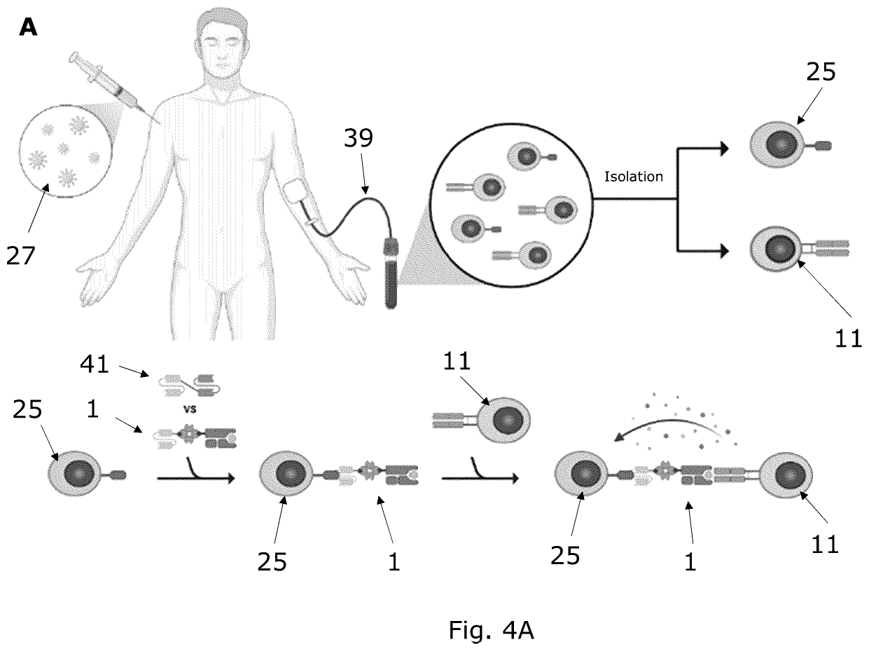

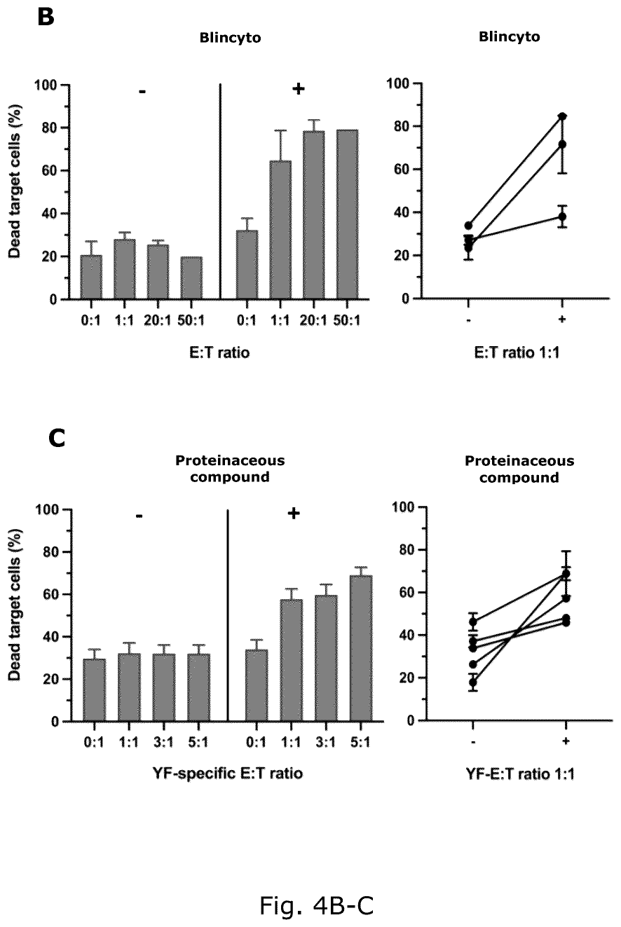

To assess the capacity of the proteinaceous compound to mediate killing of target cells by redirection of vaccine-induced YF-specific CD8+ T cells, in vitro cell killing assays using autologous cells from YF-17D-vaccinated study participants were performed and compared to the FDA-approved BiTE, Blincyto (blinatumomab, Amgen). An overview of the setup is illustrated in FIG. 4A.

As illustrated in the overview a subject is vaccinated with e.g. YFV vaccine 27 and following a blood sample 39 is obtained comprising both target cells 25 expressing the target of interest as well as YF-specific CD8+ T cells (effector cells) 11. The target cells 25 are mixed with either BiTE 41 or a proteinaceous compound 1, which binds to the target of interest (illustrated for proteinaceous compound alone). Effector cells 11 are mixed to the target cells resulting in engagement of the target cell 25 and effector cell 11 mediated by BiTE or proteinaceous compound 1 (illustrated for the proteinaceous compound alone).

In this setup, to enable tracking of the target cell population by flow cytometry, target B cells or CD4+ T cells were stained with a nontoxic, intracellular fluorescent dye, Celltrace, prior to exposure to the proteinaceous compound and autologous CD8+ T cells in varying YF-specific CD8+ T cells to target ratios. The proteinaceous compound or Blincyto-mediated killing of Celltrace+ target cells was determined with a live/dead stain.

Intriguingly, using the proteinaceous compound, the vaccine-induced YF epitope-specific effector response was redirected to target B cells achieving 26% killing at E:T 1:1 and 37% E:T 5:1 (FIG. 4C). In contrast, no target cell killing was observed in killing assays without exposure to proteinaceous compound or upon exposure to a proteinaceous compound molecule lacking an HLA-I domain. In killing assays using the FDA-approved BiTE, Blincyto (FIG. 4B), which consists of two distinct scFv domains targeting CD19 on B target cells and CD3+ T cells, pronounced killing of Celltrace+ target cells was observed achieving 37%, 53% and 59% target killing at E:T 1:1, 20:1 and 50:1, respectively.

In proteinaceous compound-mediated killing assays, target cell death is dependent on the presence of proteinaceous compound as an increase in target cell killing is only observed upon exposure to the proteinaceous compound. If no proteinaceous compound is added to the assay, no increase in target cell killing is observed with increasing numbers of effector cells added to the assay compared to the baseline level of target cell death without the presence of effector cells.

### Conclusion

Thus, in conclusion, the proteinaceous compound construct is able to both recruit and activate YF-specific CD8+ T cells leading to target cell killing.

### Example 6—Proteinaceous Compound-Mediated Killing is Dependent on Vaccine Epitope-Specific Cytotoxic T Cells

### Aim of Study

To investigate the dependence of the proteinaceous compound-mediated killing on vaccine-induced YF epitope-specific cytotoxic T cells.

### Material and Methods

See example 2.

### Results

Since a pool of total CD8+ cells obtained from YF-17D-vaccinated study participants and not only YF epitope-specific CD8+ T cells were used in in vitro target cell killing assays, the dependence of the proteinaceous compound-mediated killing on vaccine-induced YF epitope-specific cytotoxic T cells was investigated. To do this, the capacity of the proteinaceous compound to mediate killing of target B cells was assessed upon exposure to autologous CD8+ T cells obtained prior to vaccination, when no YF-specific CD8+ T cells were present (pre), or 21+/−3 days post vaccination when an YF-specific CD8+ T cell response had been induced (post). The proteinaceous compound targets CD19 and comprises a pMHC being HLA-A2-NS4B.

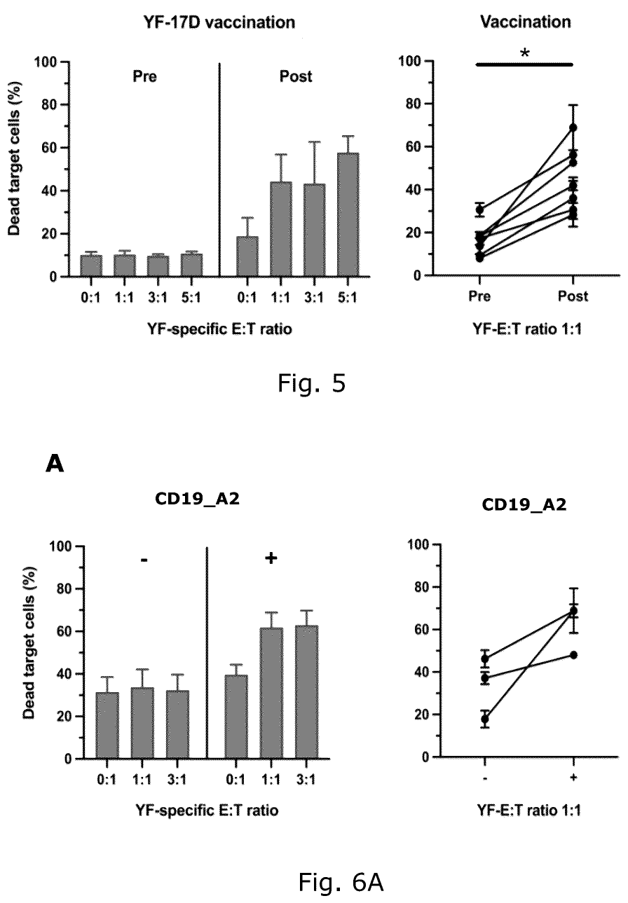

A pronounced increase of 28% in the proteinaceous compound-mediated target cell killing was obtained at YF-specific effector to target ratio (YF-E:T) 1:1 upon exposure to CD8+ T effector cells from study participants obtained post vaccination. This increase was even higher with increasing numbers of effector cells added to the assay reaching 39% target cell death at YF-E:T 5:1. In contrast, no increase in target cell killing was observed compared to background target cell death using autologous CD8+ T cells obtained from study participants prior to vaccination (FIG. 5).

### Conclusion

Thus, the proteinaceous compound-mediated killing of target cells was shown to not only be dependent on the presence of the proteinaceous compound but was also reliant on the presence of YF-specific CD8+ T cells, as an increase in target cell killing is only observed when the target cells are exposed to CD8+ T cells obtained post vaccination and thus after induction of a YF-specific effector response.

### Example 7—Proteinaceous Compound-Mediated Target Cell Killing can be Achieved Using Various HLA-I Molecules and Vaccine Epitopes

### Aim of Study

To investigate the applicability of the proteinaceous compound technology to various HLA-types and YF epitopes.

### Material and Methods

See example 2.

### Results

The HLA system is an important part of the immune defense against foreign pathogens. It is a highly complex system that varies between individuals and with distinct immunogenic vaccine epitopes presented by different HLA-I molecules.

Thus, to investigate the clinical application of the proteinaceous compound technology, it was explored whether the proteinaceous compound technology was applicable to various HLA-types and YF epitopes. This was accomplished by in vitro target cell killing assays using proteinaceous compounds comprising different HLA-I domains and immunogenic YF peptides and effector cells from study participants with HLA-types matching the proteinaceous compound HLA-domain.

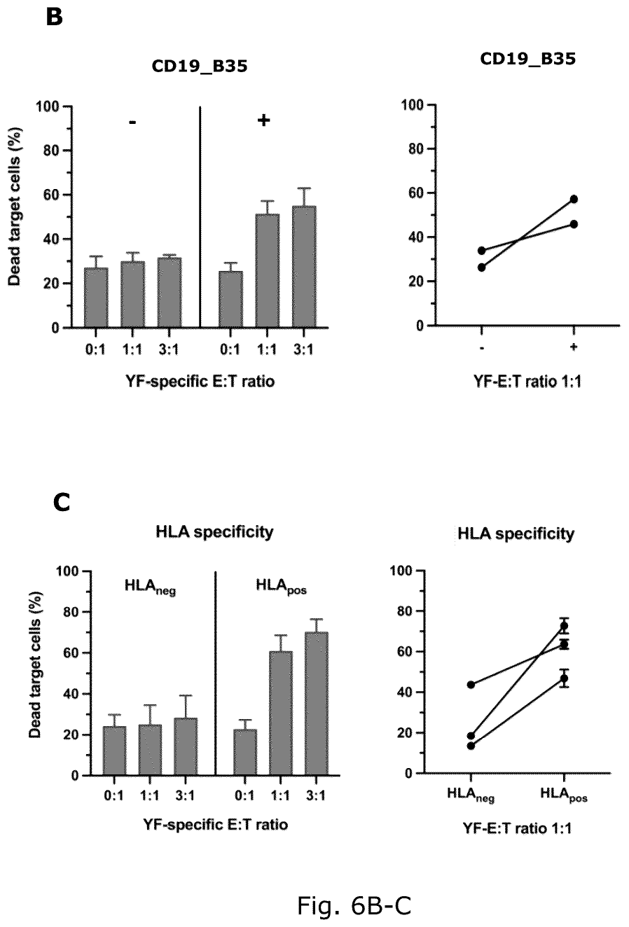

Interestingly, target cell killing could be achieved using different proteinaceous compounds targeting CD19 and comprising either NS4B-HLA-A*02:01 or NS2B-HLA-B*35:01 with specific killing of 28% and 22%, respectively, of target B cells at YF-E:T 1:1 compared to background target cell death. Thus, the proteinaceous compound-mediated target cell killing could be achieved using different HLA-molecules and epitopes, which is a prerequisite for broad application in patients with varying HLA-types. Although target cell killing could be achieved using the proteinaceous compound constructs comprising various HLA-I molecules, some YF epitopes presented on specific HLA-I molecules may be more immunogenic than others (FIG. 6A-B).

Moreover, to confirm that target cell killing was specific to the HLA-I domain within the proteinaceous compound and the HLA-type of the donor, HLA-specificity of the proteinaceous compound-mediated killing was tested in in vitro cell killing assays using the proteinaceous compounds containing HLA-I domains matching (HLAₚₒₛ) or different from (HLA_{neg}) the HLA-type of the donor (FIG. 6C).

In this setup, donors with different HLA-types (HLA-A2+, HLA-B35−, and HLA-A1+, HLA-A2−) were used to account for differences in immunogeniticy of YF peptides presented by different HLA-I molecules. For HLA-A2+, HLA-B35-individuals, a proteinaceous compound (CD19_A2) comprising a NS4B-HLA-A*02:01 domain was used as a matching proteinaceous compoundₚₒₛ, whereas the proteinaceous compound (CD19_B35) comprising a NS2B-B*35:01 was used as an unspecific proteinaceous compound_{neg}. Likewise, a proteinaceous compound (CD19_A1) comprising a NS5-A*01:01 domain and a proteinaceous compound (CD19_A2) were used as the proteinaceous compoundₚₒₛ and the proteinaceous compound_{neg}, respectively, for HLA-A1+, HLA-A2− individuals (FIG. 6C).

No target cell killing was observed using a proteinaceous compound with a HLA-domain different from the HLA-type of the donor, whereas a marked increase in target cell killing of 36% at YF-E:T 1:1 and 42% at YF-E:T 3:1 were achieved using a proteinaceous compound matching the donor's HLA-type (FIG. 6C).

### Conclusion

Thus, the proteinaceous compound-mediated target cell killing was specific for the HLA-type of the donor and could be achieved using different HLA-I molecules and YF epitopes. This is important for the clinical application of the proteinaceous compound technology as it can be adapted to be used by individuals with various HLA-types.

### Example 8—Cytokine Release

### Aim of Study

To investigate the level of cytokines in the killing assay.

### Material and Methods

See example 2.

### Results

The level of cytokines released from the proteinaceous compound-mediated specific killing of target cells was compared to killing assays with the FDA-approved BiTE, Blincyto. This was accomplished by analyzing supernatants harvested from in vitro cell killing assays by mesoscale multiplex assay for antiviral IFN-γ and inflammatory TNF-α release for proteinaceous compounds targeting CD19 and comprising pMHC being HLA-A2-NS4B.

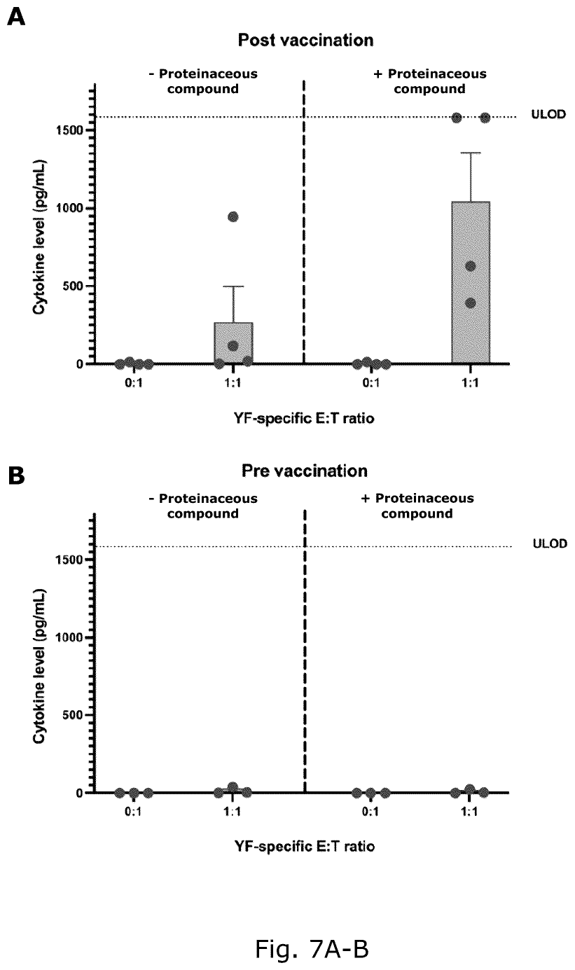

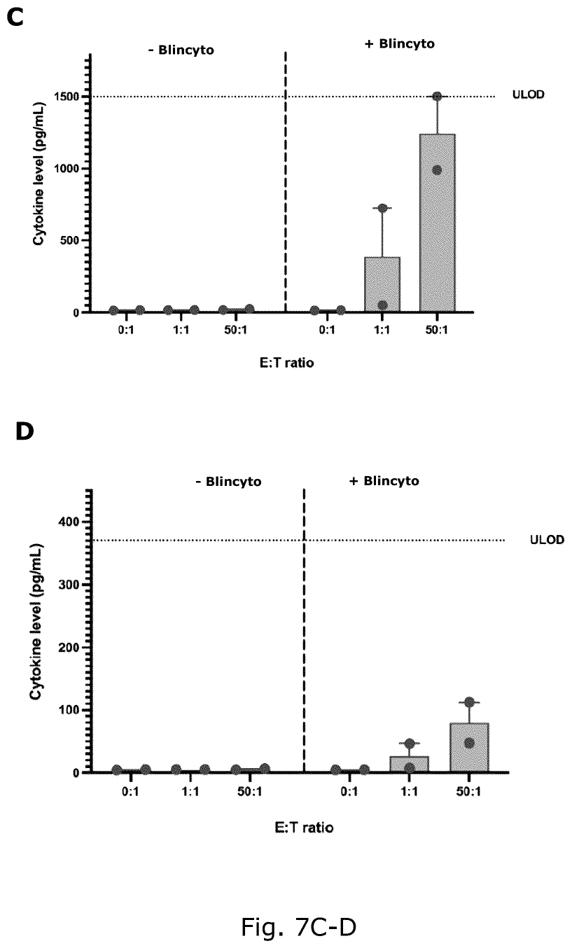

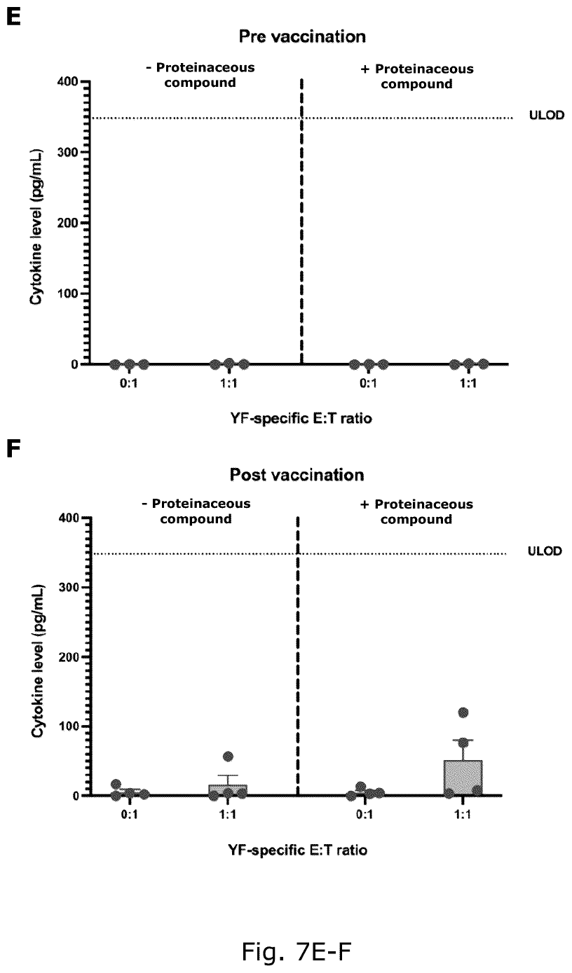

The proteinaceous compound-mediated killing of target cells was associated with pronounced release of IFN-γ (1044 pg/mL at YF-E:T 1:1) (FIG. 7A) in accordance with the induction of a cytotoxic T cell response towards target cells. In contrast, no release of IFN-γ was observed using CD8+ T cells obtained prior to YF-17D vaccination, when the proteinaceous compound-mediated killing is absent (FIG. 7B).

Surprisingly, a small increase in IFN-γ release of 270 μg/mL was observed in killing assays using CD8+ T cells post vaccination but without exposure to the proteinaceous compound. This may be an YF-17D vaccine-induced effect as no IFN-γ release is observed prior to vaccination or for Blincyto killing assays in which PBMCs from healthy donors not vaccinated with the YF-17D vaccine were used. As observed with the proteinaceous compound construct, target cell killing was associated with a pronounced increase in IFN-γ release (387 pg/mL and 1245 μg/mL at E:T 1:1 and 50:1, respectively) in in vitro cell killing assays using Blincyto (FIG. 7C).

Target cell killing was additionally associated with small increases in TNF-α release in target cell killing assays using Blincyto or the proteinaceous compound. More specifically, TNF-α levels of 27 μg/mL and 80 μg/mL were measured at E:T 1:1 and 50:1, respectively, in killing assays using Blincyto (FIG. 7D), whereas the proteinaceous compound-mediated target cell killing was associated with TNF-α release of 52 μg/mL at YF-E:T ratio 1:1 only when CD8+ T cells obtained post vaccination were used (FIG. 7E-F).

### Conclusion

The results demonstrated pronounced release of IFN-γ and increases in TNF-α release in target cell killing assays using the proteinaceous compound.

### Example 9—HIV Envelope Target

### Aim of Study

To study the adaptability of the proteinaceous compound to other targets of interest

### Material and Methods

See example 2.

### Results

To investigate the capacity of YF-specific CD8+ T cells to kill various targets, killing assays aiming a redirecting YF-specific CD8+ T cells towards HIV-1 Env-expressing cells were performed using a proteinaceous compound (scFv-10-1074) targeting HIV-1 Env and comprising a pMHC being HLA-A2-NS4B.

In this setup, both a Raji-Env cell line expressing HIV-1 envelope and GFP, and autologous CD4+ cells ex vivo infected with a full-length HIV-1-eGFP reporter virus that enables tracking of HIV-1-infected cells by GFP signaling were used as target cells. In the autologous setup, HIV-1-eGFP+ cells were enriched by sorting.

The proteinaceous compound-mediated killing of HIV-1-infected or HIV-1 Env-expressing cells by YF-specific cells could thus be assessed by the killing or disappearance of GFP+ cells compared to a control without the exposure to the proteinaceous compound or using a proteinaceous compound construct lacking the HLA-domain.

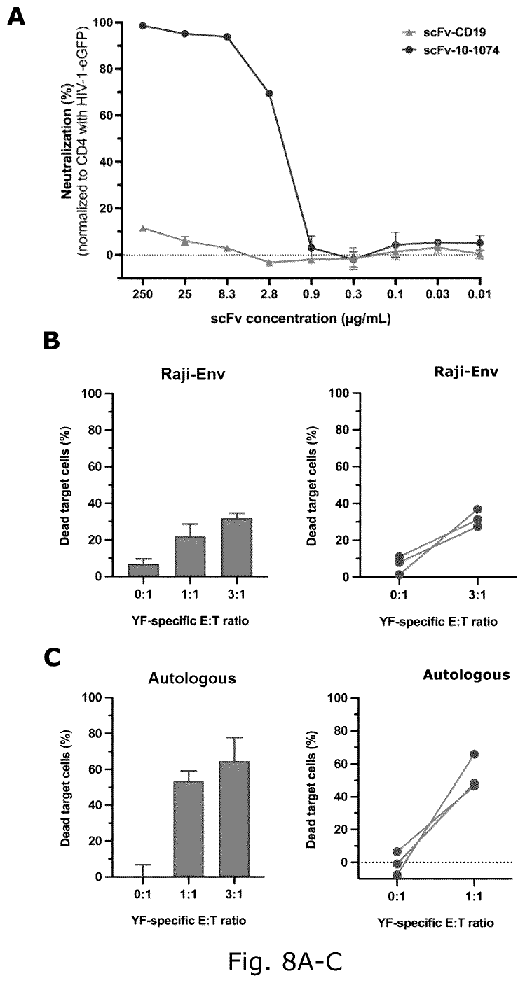

To assess the functional efficacy of the scFv-10-1074 used in the assembly of the proteinaceous compound (10-1074) construct, assays examining the capacity of the scFv-10-1074 to bind HIV-1-infected cells (FIG. 2B) and neutralize free HIV-1 (FIG. 8A) to prevent infection of CD4+ T cells were carried out. The scFv-10-1074 used in the assembly of the proteinaceous compound (10-1074) were able to neutralize full-length HIV-1-eGFP and prevent infection of CD4+ T cells (FIG. 8A). This scFv-10-1074 neutralization of HIV-1-eGFP was dose-dependent and envelope-specific as a scFv-CD19 was unable to neutralize the virus.

Using a Raji-Env or CD4+ T cells ex vivo infected with the full length HIV-1-eGFP reporter virus, we additionally showed that the scFv-10-1074 was able to bind HIV-1 Env expressing target cells, thereby verifying both the HIV-1-eGFP reporter virus and functionality of scFv-10-1074 used in the proteinaceous compound (10-1074) construct.

Using the proteinaceous compound (10-1074) construct, we were able to redirect the vaccine-induced YF epitope-specific effector response to target Raji-Env cells achieving 22% and 32% killing at YF-E:T 1:1 and 3:1, respectively (FIG. 8B). Importantly, when infecting autologous CD4+ T cells with full-length HIV-1-eGFP, the proteinaceous compound-linked vaccine-induced CD8+ T cells were able to mediate specific killing of 54% at YF-E:T 1:1 and 65% target killing at YF-E:T of 3:1 (FIG. 8C). This is in a range comparable to Blincyto-mediated killing of B cells with 65% killing at E:T 1:1.

A major obstacle of HIV-1 infection is viral immune escape due to a high mutation rate of HIV-1. By designing the proteinaceous compound against HIV-1 infected cells using a scFv domain from broadly neutralizing antibodies targeting conserved regions or by using combinations of different scFv domains, we may be able to reduce viral escape from the proteinaceous compound. Moreover, by binding directly to the HIV-1 Env on target cells, the proteinaceous compound technology may be able to overcome the obstacle of HLA downregulation, which is a trait by HIV-infected cells.

### Conclusion

Specific killing of various targets demonstrate that the proteinaceous compound technology is easily adapted to recognize any cell surface antigen of interest by changing the scFv domain of the proteinaceous compound and thus holds great promise for various diseases.

### Example 10—CD4+ T Cells

### Aim of Study

To investigate the capacity of YF-specific CD8+ T cells to kill various targets, killing assays aiming a redirecting YF-specific CD8+ T cells towards CD4+ T target cells were performed.

### Material and Methods

A proteinaceous compound (CD4) construct assembled from a biotin-conjugated anti-CD4 antibody (BioLegend, #344610) linked via streptavidin to a biotin-conjugated HLA-I molecule presenting an immunogenic YF peptide (HLA-A2 NS4B).

The ‘In vitro cell killing’ was performed similar to the method described under example 2.

### Results

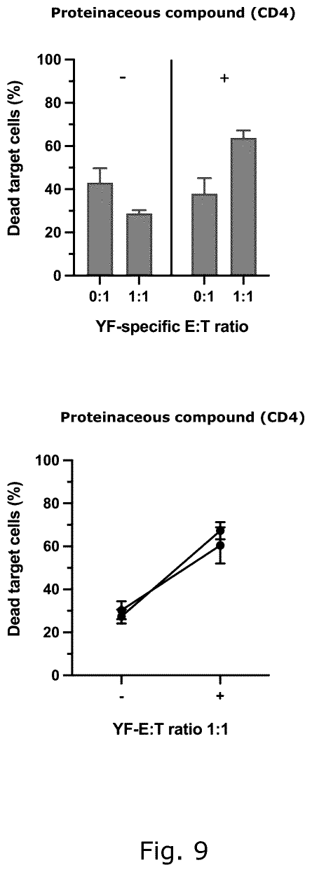

In this setup, CD4+ target cells were exposed to autologous CD8+ T cells from YF-17D vaccinated study participants in the presence of the proteinaceous compound (CD4) at a YF-specific E:T ratio of 1:1. Using the proteinaceous compound (CD4) the vaccine-induced YF epitope-specific effector response was redirected to target CD4+ T cells achieving 35% killing at YF-E:T 1:1 compared to a negative control without exposure to the proteinaceous compound (CD4) construct (FIG. 9).

### Conclusion

The proteinaceous compound technology is easily adapted to recognize cell surface antigen of CD4+ by changing the antigen-binding domain of the proteinaceous compound.

### Example 11—Production of Optimized Proteinaceous Compounds

### Aim of Study

To produce optimized proteinaceous compounds using Knob-into-Hole technique.

### Material and Methods

Expi293F cells were, in the presence of biotin, co-transfected with two plasmids encoding either HLA-A2 heavy chain-IgG hole (SEQ ID NO: 21) or Beta₂ microglobulin-NS4B-IgG knob (SEQ ID NO: 20) to produce recombinant biotinylated HLA-A2-NS4B monomers. The assembled recombinant HLA-A2 NS4B-biotin monomers without binding moieties were purified from cell supernatant on day 5 post-transfection using a Ni²⁺-NTA column.

To test the functionality of the recombinant HLA-A2 Yellow Fever (YF) NS4B Knob-into-Hole protein, the monomer was tetramerized (YFV Tetramer-PE) and incubated with PBMCs from an HLA-A2 positive YF vaccinated donor or an HLA-A2 negative donor similar to methods described in example 2. Tetramerization was obtained by three sequential additions of biotinylated YF peptide-HLA class I monomers to Streptavidin-PE in a 4:1 molar ratio, each addition followed by an incubation step at 4° C. for 15 minutes according to the manufacturer's instructions.

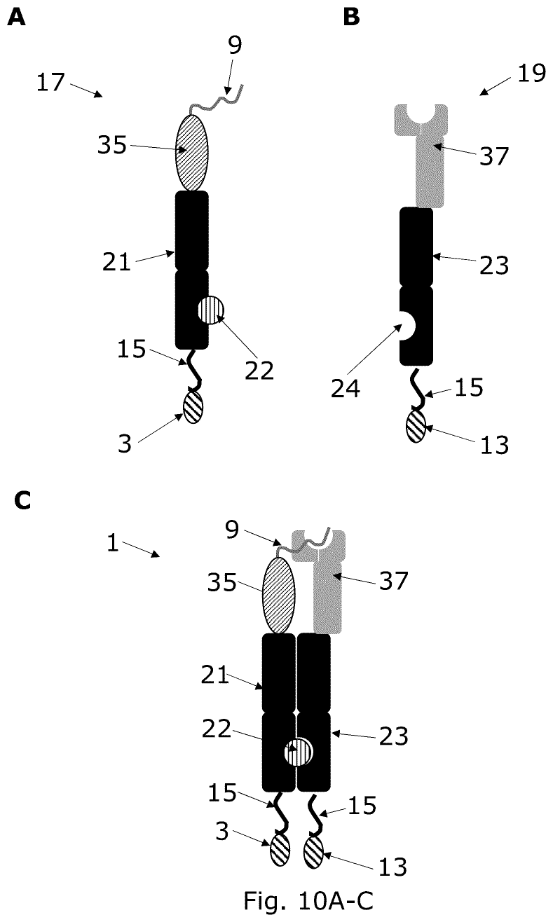

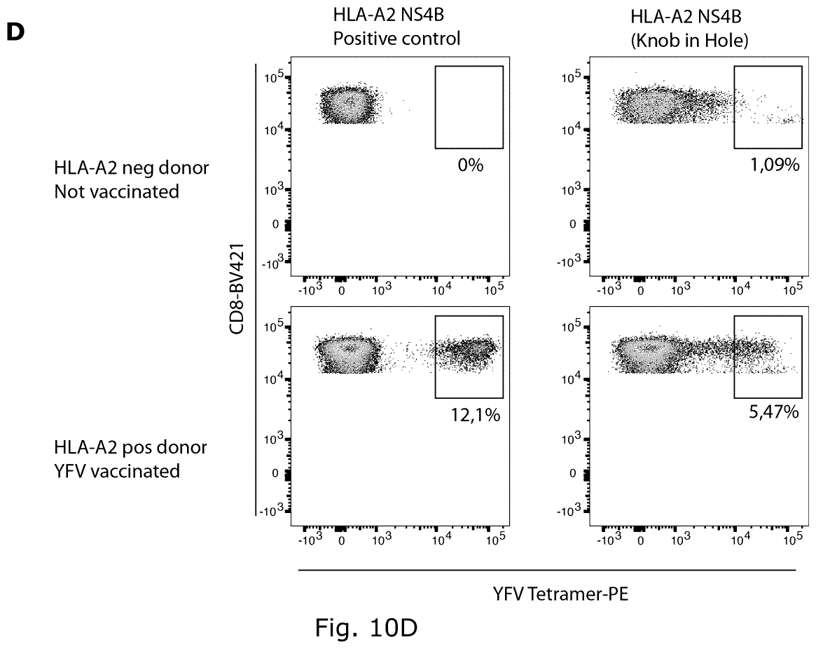

As a positive control the commercially available HLA-A2 NS4B were used (upper and lower left flow plots in FIG. 10D). HLA-A2 NS4B monomer from Immunaware (ImmunAware, #1002-09) was tetramerized using PE-conjugated streptavidin (BioLegend, #405204).

### Results

An illustration of the Knob-into-Hole technique is illustrated in FIG. 10A-C. FIG. 10A illustrates a first subunit 17 of the proteinaceous compound, while FIG. 10B illustrates a second subunit 19 of the proteinaceous compound. FIG. 10C illustrates the first and second subunit 17,19 being combined to form a functional proteinaceous compound 1. The first subunit 17 includes an antigenic peptide 9, β2-microglobulin 35, a knob part 21 comprising a knob 22 and a linker 15 connecting said knob part 21 with said first binding moiety 3. The second subunit 19 includes the second part of the MHC molecule i.e. the HLA molecule without the β2-microglobulin, a hole part 23 with a hole 24, and a linker 15 connecting a second binding moiety 13 to the hole part. FIG. 10C illustrates how the knob 22 in the knob part 21 integrates with the hole 24 in the hole part 23.

The recombinant HLA-A2 NS4B Knob-into-Hole protein shows 5.47% NS4B specific CD8 cells (lower right flow plot) compared to a negative donor 1.09% (upper right flow plot in FIG. 10D). A positive control using HLA-A2 NS4B shows 12.1% NS4B specific CD8 cells (lower left flow plot) compared to a HLA-A2 negative donor 0% (upper left flow plot) compared to the staining achieved by the HLA-A2 NS4B Knob-into-Hole protein.

### Conclusion

The Knob-into-Hole technique can be used for designing functional proteinaceous compounds.

### REFERENCES

Altschul S F, Gish W, Miller W, Myers E W, Lipman D J: Basic local alignment search tool. J Mol Biol 1990; 215(3): 403-410.
Dufloo J, et al.: Anti-HIV-1 antibodies trigger non-lytic complement deposition on infected cells. EMBO reports 2020, 21: e49351.
Serra P, et al.: Increased yields and biological potency of knob-into-hole-based soluble MHC class II molecules. Nature Communications 2019; 10: 4917.

### REFERENCE NUMBERS

1: proteinaceous compound
3: first binding moiety
5: target of interest
7: MHC molecule
9: antigenic peptide
11: CD8+ T-cells
13: second binding moiety
15: linker
17: first subunit
19: second subunit
21: knob part
22: knob
23: hole part
24: hole
25: target cell
27: attenuated YFV
29: T-cell receptor
31: perforin granzymes
33: IFN-γ; TNF-α
35: β2-microglobulin
37: MHC molecule minus β2-microglobulin
39: blood sample
41: BiTE

### SEQUENCE LISTING

SEQ ID NO: 1—Linker 1
SEQ ID NO: 2—Linker 2
SEQ ID NO: 3—Linker 3
SEQ ID NO:4—Knob part
SEQ ID NO: 5—Hole part
SEQ ID NO: 6—Antigenic peptide—NS5
SEQ ID NO: 7—Antigenic peptide—NS4B
SEQ ID NO: 8—Antigenic peptide—NS3
SEQ ID NO: 9—Antigenic peptide—NS3
SEQ ID NO: 10—Antigenic peptide—NS5
SEQ ID NO: 11—Antigenic peptide—NS3
SEQ ID NO: 12—Antigenic peptide—NS2A
SEQ ID NO: 13—Antigenic peptide—NS2B
SEQ ID NO: 14—Antigenic peptide—E
SEQ ID NO: 15—Antigenic peptide—NS1
SEQ ID NO: 16—binding moiety scFv—CD19
SEQ ID NO: 17—binding moiety scFV—10-1074
SEQ ID NO: 18—scFv CD19 in pcDNA3.1
SEQ ID NO: 19—scFv 10-1074 in pcDNA3.1
SEQ ID NO: 20—Knob-into-Hole (no ScFv)—Knob
SEQ ID NO: 21—Knob-into-Hole (no ScFv)—Hole
SEQ ID NO: 22—Knob-into-Hole—Knob1
SEQ ID NO: 23—Knob-into-Hole design—Knob2
SEQ ID NO: 24—Knob-into-Hole design—Knob3
SEQ ID NO: 25—Knob-into-Hole design—Hole1
SEQ ID NO: 26—Knob-into-Hole design—Hole2
SEQ ID NO: 27—Knob-into-Hole design—Hole3
SEQ ID NO: 28—pBR-HIV-1 M NL4-3 R5
SEQ ID NO: 29—encoded CD19 scFv with leader and Avitag from pcDNA3.1
SEQ ID NO: 30—encoded 10-1074 scFV with leader and Avitag from pcDNA3.1

1. A proteinaceous compound comprising:
a) a first binding moiety, having affinity for a target of interest, and
b) a pMHC comprising an MHC molecule, presenting an antigenic peptide, wherein the presented antigenic peptide has affinity for CD8+ T-cells induced by the antigenic peptide, wherein β2-microglobulin is non-covalently associated.

2-32. (canceled)

33. The proteinaceous compound according to claim 1, wherein a first subunit comprises said antigenic peptide of the pMHC, the β2-microglobulin and said first binding moiety.

34. The proteinaceous compound according to claim 1, wherein the antigenic peptide is derived from a virus.

35. The proteinaceous compound according to claim 1, wherein the antigenic peptide is derived from Yellow Fever Virus (YFV), measles, rubella, varicella or smallpox.

36. The proteinaceous compound according to claim 1, wherein the antigenic peptide comprises or consists of an epitope selected from the group consisting of SEQ ID NO: 7, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14 and SEQ ID NO: 15.

37. The proteinaceous compound according to claim 1, comprising a second binding moiety having affinity for a target of interest.

38. The proteinaceous compound according to claim 1, wherein a first subunit comprises said antigenic peptide of the pMHC, the β2-microglobulin and said first binding moiety; and a second subunit comprises the MHC molecule without the β2-microglobulin and a second binding moiety, having affinity for a target of interest.

39. The proteinaceous compound according to claim 1, wherein a first subunit comprises said antigenic peptide of the pMHC, the β2-microglobulin and said first binding moiety; and a second subunit comprises the MHC molecule without the β2-microglobulin and a second binding moiety, having affinity for a target of interest wherein the first binding moiety and second binding moiety have affinity for different targets of interest.

40. The proteinaceous compound according to claim 1, wherein a first subunit comprises said antigenic peptide of the pMHC, the β2-microglobulin and said first binding moiety; and a second subunit comprises the MHC molecule without the β2-microglobulin and a second binding moiety, having affinity for a target of interest said first subunit and said second subunit being linked via a heterodimerization domain.

41. The proteinaceous compound according to claim 1, wherein a first subunit comprises said antigenic peptide of the pMHC, the β2-microglobulin and said first binding moiety; and a second subunit comprises the MHC molecule without the β2-microglobulin and a second binding moiety, having affinity for a target of interest said first subunit and said second subunit being linked via a heterodimerization domain selected from the group consisting of a Knob-into-Hole system, c-jun transcription factor derived dimerization domains, and leucine zipper dimerization domains.

42. The proteinaceous compound according to claim 1, wherein the binding moiety is selected from the group consisting of polyclonal antibody, a monoclonal antibody, an antibody wherein the heavy chain and the light chain are connected by a flexible linker, an Fv molecule, an antigen binding fragment, a Fab fragment, a Fab′ fragment, a F(ab′)2 molecule, a fully human antibody, a humanized antibody, a chimeric antibody, scFv (single-chain variable fragment) and a single-domain antibody (sdAb) (nanobody or diabody), preferably a scFv (single-chain variable fragment) and a single-domain antibody (sdAb) (nanobody or diabody).

43. The proteinaceous compound according to claim 1, wherein the binding moiety having affinity for a target of interest, has affinity for
an intracellular pathogen; or
a cancer cell surface protein.

44. The proteinaceous compound according to claim 1, wherein the binding moiety having affinity for a target of interest, has affinity for
immunodeficiency virus (HIV), viral hepatitis or human T-cell lymphotropic virus type 1 (HTLV); or
CD19, CD4, CD20, GD2, PSMA, or Mesothelin.

45. The proteinaceous compound according to claim 1, wherein the proteinaceous compound is in a composition.

46. A method of treating a subject infected with an intracellular pathogen or suffering from cancer, the method comprising administering the proteinaceous compound according to claim 1, wherein the subject has previously received a vaccine for inducing CD8+ T-cells directed against the antigenic peptide;
wherein the binding moiety, having affinity for a target of interest, has affinity for an epitope of the pathogen causing the disease or a cell surface marker of the cancer.

47. A nucleic acid encoding a proteinaceous compound comprising:
a) a first binding moiety, having affinity for a target of interest, and
b) a pMHC comprising an MHC molecule, presenting an antigenic peptide,
wherein the presented antigenic peptide has affinity for CD8+ T-cells induced by the antigenic peptide, wherein β2-microglobulin is non-covalently associated.

48. The nucleic acid according to claim 47, wherein the nucleic acid is comprised in a vector.

49. The nucleic acid according to claim 47, wherein the nucleic acid is in the form of a plasmid.

50. The nucleic acid according to claim 47, wherein the nucleic acid is comprised a cell.

51. The nucleic acid according to claim 47, wherein the nucleic acid is comprised in a mammalian cell.
